SEQUENCE LISTING (11 sequences)

SEQ ID NO: 1 - DNA, 3183 bp; single-stranded, linear
cctccaggccccgccgcgcgtcccgggggccggccccgcgagcgcaggagtaaacaccgccggagtcttggagccgctgcagaagggaataaagagagatgcagggatttgtgaggttacggcgccccagctgcaagatgcactagccggctgaacccgggatcggctgacttgttggaaccggagtgctctgcacggagagtggtggatgagttgaagttgccttcccggggctcattttccacgctgccgagaggaatccgagaggcaaggcaatcacttcgtcttgccattgattgggtatcgggagctttttttttctcccctctctctttcttttcctccgtcttgttgcatgcaagaaaattacagtccgctgctcgcccgccctgggtgcgagatattcagccccgctctctcccgtgcattgtgcaacccaaagatgaaagaccgaaggggagaaagttaaagaaatcgcccacatgcgctggatcagtccacggcttggggaaaggcatccagagaaggtgggagcggagagtttgaagtctttacaggcgggaagatggcggactggagctgaaagtgttgattgggaaacttgggtgattcttgtgtttatttacaatcctcttgacccaggcaggacacatgcaggccaaaaaacgctatttcatcctgctctcagctggctcttgtctcgcccttttgttttatttcggaggcttgcagtttagggcatcgaggagccacagccggagagaagaacacagcggtaggaatggcttgcaccaccccagtccggatcatttctggccccgcttcccggagcctctgcgccccttcgttccttgggatcaattggaaaacgaggattccagcgtgcacatttccccccggcagaagcgagatgccaactccagcatctacaaaggcaagaagtgccgcatggagtcctgcttcgatttcaccctttgcaagaaaaacggcttcaaagtctacgtatacccacagcaaaaaggggagaaaatcgccgaaagttaccaaaacattctagcggccatcgagggctccaggttctacacctcggaccccagccaggcgtgcctctttgtcctgagtctggatactttagacagagaccagttgtcacctcagtatgtgcacaatttgagatccaaagtgcagagtctccacttgtggaacaatggtaggaatcatttaatttttaatttatattccggcacttggcctgactacaccgaggacgtggggtttgacatcggccaggcgatgctggccaaagccagcatcagtactgaaaacttccgacccaactttgatgtttctattcccctcttttctaaggatcatcccaggacaggaggggagagggggtttttgaagttcaacaccatccctcctctcaggaagtacatgctggtattcaaggggaagaggtacctgacagggataggatcagacaccaggaatgccttatatcacgtccataacggggaggacgttgtgctcctcaccacctgcaagcatggcaaagactggcaaaagcacaaggattctcgctgtgacagagacaacaccgagtatgagaagtatgattatcgggaaatgctgcacaatgccactttctgtctggttcctcgtggtcgcaggcttgggtccttcagattcctggaggctttgcaggctgcctgcgtccctgtgatgctcagcaatggatgggagttgccattctctgaagtgattaattggaaccaagctgccgtcataggcgatgagagattgttattacagattccttctacaatcaggtctattcatcaggataaaatcctagcacttagacagcagacacaattcttgtgggaggcttatttttcttcagttgagaagattgtattaactacactagagattattcaggacagaatattcaagcacatatcacgtaacagtttaatatggaacaaacatcctggaggattgttcgtactaccacagtattcatcttatctgggagattttccttactactatgctaatttaggtttaaagcccccctccaaattcactgcagtcatccatgcggtgacccccctggtctctcagtcccagccagtgttgaagcttctcgtggctgcagccaagtcccagtactgtgcccagatcatagttctatggaattgtgacaagcccctaccagccaaacaccgctggcctgccactgctgtgcctgtcgtcgtcattgaaggagagagcaaggttatgagcagccgttttctgccctacgacaacatcatcacagacgccgtgctcagccttgacgaggacacggtgctttcaacaacagaggtggatttcgccttcacagtgtggcagagcttccctgagaggattgtggggtaccccgcgcgcagccacttctgggataactctaaggagcggtggggatacacatcaaagtggacgaacgactactccatggtgttgacaggagctgctatttaccacaaatattatcactacctatactcccattacctgccagccagcctgaagaacatggtggaccaattggccaattgtgaggacattctcatgaacttcctggtgtctgctgtgacaaaattgcctccaatcaaagtgacccagaagaagcagtataaggagacaatgatgggacagacttctcgggcttcccgttgggctgaccctgaccactttgcccagcgacagagctgcatgaatacgtttgccagctggtttggctacatgccgctgatccactctcagatgaggctcgaccccgtcctctttaaagaccaggtctctattttgaggaagaaataccgagacattgagcgactttgaggaatccggctgagtgggggaggggaagcaagaagggatgggggtcaagctgctctctcttcccagtgcagatccactcatcagcagagccagattgtgccaactatccaaaaacttagatgagcagaatgacaaaaaaaaaaaaggccaatgagaactcaactcctggctcctgggactgcaccagactgctccaaactcacctcactggcttctgtgtcccaagactaggttggtacagtttaattatggaacattaaataattatttttgaaaaaaaaaaaaaaaaaa

SEQ ID NO: 2 - protein, 746 aa
MQAKKRYFILLSAGSCLALLFYFGGLQFRASRSHSRREEHSGRNGLHHPSPDHFWPRFPEPLRPFVPWDQLENEDSSVHISPRQKRDANSSIYKGKKCRMESCFDFTLCKKNGFKVYVYPQQKGEKIAESYQNILAAIEGSRFYTSDPSQACLFVLSLDTLDRDQLSPQYVHNLRSKVQSLHLWNNGRNHLIFNLYSGTWPDYTEDVGFDIGQAMLAKASISTENFRPNFDVSIPLFSKDHPRTGGERGFLKFNTIPPLRKYMLVFKGKRYLTGIGSDTRNALYHVHNGEDVVLLTTCKHGKDWQKHKDSRCDRDNTEYEKYDYREMLHNATFCLVPRGRRLGSFRFLEALQAACVPVMLSNGWELPFSEVINWNQAAVIGDERLLLQIPSTIRSIHQDKILALRQQTQFLWEAYFSSVEKIVLTTLEIIQDRIFKHISRNSLIWNKHPGGLFVLPQYSSYLGDFPYYYANLGLKPPSKFTAVIHAVTPLVSQSQPVLKLLVAAAKSQYCAQIIVLWNCDKPLPAKHRWPATAVPVVVIEGESKVMSSRFLPYDNIITDAVLSLDEDTVLSTTEVDFAFTVWQSFPERIVGYPARSHFWDNSKERWGYTSKWTNDYSMVLTGAAIYHKYYHYLYSHYLPASLKNMVDQLANCEDILMNFLVSAVTKLPPIKVTQKKQYKETMMGQTSRASRWADPDHFAQRQSCMNTFASWFGYMPLIHSQMRLDPVLFKDQVSILRKKYRDIERL

SEQ ID NO: 3 - DNA, 3003 bp; single-stranded, linear
ctcgccagcccagactcggccctggcagtggcggctggcgattcggaccgatccgacctgggcggaggtggcccgcgccccgcggcatgagccggtgaccaagctcggggccgagcgggaggcagccgtggccgaggagtgtgaggaagaggctgtctgtgtcattatgtgtgcgtcggtcaagtataatatccggggtcctgccctcatcccaagaatgaagaccaagcaccgaatctactatatcaccctcttctccattgtcctcctgggcctcattgccactggcatgtttcagttttggccccattctatcgagtcctcaaatgactggaatgtagagaagcgcagcatccgtgatgtgccggttgttaggctgccagccgacagtcccatcccagagcggggggatctcagttgcagaatgcacacgtgttttgatgtctatcgctgtggcttcaacccaaagaacaaaatcaaggtgtatatctatgctctgaaaaagtacgtggatgactttggcgtctctgtcagcaacaccatctcccgggagtataatgaactgctcatggccatctcagacagtgactactacactgatgacatcaaccgggcctgtctgtttgttccctccatcgatgtgcttaaccagaacacactgcgcatcaaggagacagcacaagcgatggcccagctctctaggtgggatcgaggtacgaatcacctgttgttcaacatgttgcctggaggtcccccagattataacacagccctggatgtccccagagacagggccctgttggctggtggcggcttttctacgtggacttaccggcaaggctacgatgtcagcattcctgtctatagtccactgtcagctgaggtggatcttccagagaaaggaccaggtccacggcaatacttcctcctgtcatctcaggtgggtctccatcctgagtacagagaggacctagaagccctccaggtcaaacatggagagtcagtgttagtactcgataaatgcaccaacctctcagagggtgtcctttctgtccgtaagcgctgccacaagcaccaggtcttcgattacccacaggtgctacaggaggctactttctgtgtggttcttcgtggagctcggctgggccaggcagtattgagcgatgtgttacaagctggctgtgtcccggttgtcattgcagactcctatattttgcctttctctgaagttcttgactggaagagagcatctgtggttgtaccagaagaaaagatgtcagatgtgtacagtattttgcagagcatcccccaaagacagattgaagaaatgcagagacagctcttcatggaaccagtcaggagagagaactggtcagctgctaatcaccaaatgaactccctgatctggcctagggaacagtgggattcacagattatcaatgaccggatctatccatatgctgccatctcctatgaagaatggaatgaccctcctgctgtgaagtggggcagcgtgagcaatccactcttcctcccgctgatcccaccacagtctcaagggttcaccgccatagtcctcacctacgaccgagtagagagcctcttccgggtcatcactgaagtgtccaaggtgcccagtctatccaaactacttgtcgtctggaataatcagaataaaaaccctccagaagattctctctggcccaaaatccgggttccattaaaagttgtgaggactgctgaaaacaagttaagtaaccgtttcttcccttatgatgaaatcgagacagaagctgttctggccattgatgatgatatcattatgctgacctctgacgagctgcaatttggttatgaggtctggcgggaatttcctgaccggttggtgggttacccggatcgtctgcatctctgggaccatgagatgaataagtggaagtatgagtctgagtggacgaatgaagtgtccatggtgctcactggggcagctttttatcacaagtattttaattacctgtatacctacaaaatgcctggggatatcaagaactgggtagatgctcatatgaactgtgaagatattgccatgaacttcctggtggccaacgtcacgggaaaagcagttatcaaggtaaccccacgaaagaaattcaagtgtcctgagtgcacagccatagatgggctttcactagaccaaacacacatggtggagaggtcagagtgcatcaacaagtttgcttcagtcttcgggaccatgcctctcaaggtggtggaacaccgagctgaccctgtcctgtacaaagatgactttcctgagaagctgaagagcttccccaacattggcagcttatgaaacgtgtcattggtggaggtctgaatgtgaggctgggacagagggagagaacaaggcctcccagcactctgatgtcagagtagtaggttaagggtggaaggttgacctacttggatcttggcatgcacccacctaacccactttctcaagaacaagaacctagaatgaatatccaagcacctcgagctatgcaacctctgttcttgtatttcttatgatctctgatgggttcttctcgaaaatgccaagtggaagactttgtgggcatgctcccagatttaaatccagctgaggctccctttgttttcagttccatgtaacaatctggaaggaaacttcacggacaggaagactgctggagaagagaagcgtgttagcccatttgaggtctggggaatcatgtaaagggtacccagacctcacttttagttatttacatcaatgagttctttcagggaaccaaacccagaattcggtgcaaaagccaaacatcttggtgggatttgataaatgccttgggacctggagtgctgggcttgtgcacaggaagagcaccagccgctgagtcaggatcctgtcagttccatgagctattcctctttggtttggctttttgatatgattaaaattattttttattccttttaaaa

SEQ ID NO: 4 - protein, 728 aa [sequence omitted]

SEQ ID NO: 5 - DNA, 16 bp; single-stranded, linear
nnnbngucnnnnnnnn

SEQ ID NO: 6 - DNA, 46 bp; single-stranded, linear
cgggatcccgcaggacacatgcaggccaaaaaacgctatttcatcc

SEQ ID NO: 7 - DNA, 49 bp; single-stranded, linear
ttttccttttgcggccgcttttttccttaagtcgctcaatgtctcggta

SEQ ID NO: 8 - DNA, 42 bp; single-stranded, linear
cgggatcccgcacatgcagtttagggcatcgaggagccacag

SEQ ID NO: 9 - DNA, 33 bp; single-stranded, linear
tccccgcggggagattttctcccctttttgctg

SEQ ID NO: 10 - DNA, 51 bp; single-stranded, linear
caaagcctccaggaatctgaaggacccaagcctgcgatcacgaggaaccag

SEQ ID NO: 11 - DNA, 51 bp; single-stranded, linear
caaagcctccaggaatctgaaggacccaagcctgcaaccacgaggaaccag

(19) United States
(12) Patent Application Publication
(10) Pub. No.: US 2002/0156044 A1
(43) Pub. Date: Oct. 24, 2002

(54) Use of EXT genes for the treatment of cancer and other diseases

(75) Inventors: Frank Tufaro, Vancouver (CA); M. Craig McCormick, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: University of British Columbia, Vancouver (CA)

(21) Appl. No.: 10/106,092

(22) Filed: Mar. 25, 2002

Related U.S. Application Data
(63) Continuation of application No. 09/060,940, filed on Apr. 15, 1998.

(51) Int. Cl. A61K 48/00; A61K 31/727; C12N 15/86; C12N 15/861; C12N 15/867

(52) U.S. Cl. 514/44; 435/456; 514/56

(57) ABSTRACT

Methods for treating tumors are provided comprising the general step of administering to a patient a gene delivery vehicle or vector which directs the expression of an EXT gene, such that growth of the tumor is diminished.

### TECHNICAL FIELD

The present invention generally relates to pharmaceutical compositions and methods, and more specifically, to the use of EXT genes in the treatment of cancer and other gags diseases.

### BACKGROUND OF THE INVENTION

Viruses and other pathogens have evolved to exploit the vast array of proteins and carbohydrates expressed on cell surfaces to gain entry into host cells (see, e.g., Spear, P. G. “Entry of alphaherpesviruses into cells” Sem. Virol. 4:167-180, 1993; Rostand, K. S., and Esko, J. D., “Microbial adherence to and invasion through proteoglycans,” Infection & Immunity 65:1-8, 1997). Moreover, several viruses, including adenovirus, HIV, and HSV, appear to recognize more than one receptor to gain entry to suitable host cells (see, e.g., Haywood, A. M. “Virus receptors: binding, adhesion strengthening, and changes in viral structure,” J Virol 68:1-5, 1994; Tufaro, F. “Virus entry: two receptors are better than one,” Trends in Microbiology 5:257-8, 1997). In the case of the enveloped viruses, one or more glycoproteins embedded in the lipid envelope acts as an anti-receptor to bind to the host cell surface, and fusion with the plasma membrane or an endosomal membrane is mediated by a viral fusion peptide or domain. For herpes simplex virus the situation is especially complex because herpes simplex virus encodes at least 11 glycoproteins (see Spear, P. G., “Glycoproteins specified by herpes simplex viruses,” In The Herpesviruses, B. Roizman, ed. (New York: Plenum Publishing Corp), pp. 315-356, 1985).

Although substantial progress is being made in identifying the glycoproteins that influence virus attachment and entry, less is known about how these determinants recognize and interact with the cell surface components. In the case of Herpes Simplex Virus, the current model for HSV infection predicts that a spike protein, probably gC or gB, embedded in the envelope of the virion contacts glycosaminoglycan moieties on cell surface proteoglycans, which are ubiquitous components of mammalian cells and tissues. Sulfated glycosaminoglycans including heparin/heparan sulfate (HS), chondroitin sulfate (CS) and dermatan sulfate (DS) are covalently bound to Ser residues in the core proteins through the common carbohydrate-protein linkage structure, GlcA-Gal-Gal-Xyl-O-Ser. Heparan sulfate is synthesized when GlcNAc is added to this linker region, whereas chondroitin sulfate is formed after GalNAc addition.

As described in more detail below, it has now been discovered that EXT genes, which are presumed to be involved in the human disease hereditary multiple exostoses (HME), have profound effects on the susceptibility of host cells to HSV infection, and moreover, provides a foundation which establishes the role of proteoglycans in the development of cancer.

Briefly, cancer accounts for one-fifth of the total mortality in the United States, and is the second leading cause of death. Cancer is typically characterized by the uncontrolled division of a population of cells. This uncontrolled division typically leads to the formation of a tumor, which may subsequently metastasize to other sites.

Primary solid tumors can generally be treated by surgical resection. However, the majority of patients which have solid tumors also possess micrometastases beyond the primary tumor site. If treated with surgery alone, approximately 70% of these patients will experience recurrence of the cancer. In addition to surgery, many cancers are now also treated with a combination of therapies involving cytotoxic chemotherapeutic drugs (e.g., vincristine, vinblastine, cisplatin, methotrexate, 5-FU, etc.) and/or radiation therapy. One difficulty with this approach however, is that radiotherapeutic and chemotherapeutic agents are toxic to normal tissues, and often create life-threatening side effects. In addition, these approaches often have extremely high failure/remission rates (up to 90% depending upon the type of cancer).

One tumor (which may degenerate into a cancer), is Hereditary multiple exostoses (HME), which is an autosomal dominant disorder characterized by the formation of cartilage-capped tumors (exostoses) that develop from the growth plate of endochondral bone (Solomon, J. Bone Joint Surg. 45:292-304, 1963). This condition can lead to skeletal abnormalities, short stature, and in some instances, malignant transformation from exostoses to chondrosarcomas (J. Med. Genet. 28:262-266, 1991); Leone et al., Journal of Heredity 78:171-177, 1987) or osteosarcomas (Schmale et al., J. Bone Joint Surg. Am. 76:986-992, 1994); Luckert-Wicklund et al., Am. J. Med. Genet. 55:43-46, 1995).

It is apparent that there exists a need for new and additional methods and compositions which address and rectify the problem. The present invention fulfills this need, and further provides related advantages.

### SUMMARY OF THE INVENTION

Briefly stated, the present invention provides methods for altering or changing glycosaminoglycan (e.g., heparin or heparan-sulfate, chondrotin or chondrotin sulfate, keratin sulfate, dermitan sulfate, or hyaluronic acid) expression and/or activity in cells (i.e., either up-regulation or down-regulation) comprising the general step of altering expression of an EXT gene in the cells, and thereby altering glycosaminoglycan expression. As utilized within the context of the present invention, the expression of an EXT gene is “altered” or “changed” if the quantity or species of glycosaminoglycan which is expressed on the surface of the cell is increased or decreased in a statistically significant manner. In certain instances, this can be greater than a 25% or 100% change in either the quantity or species of glycosaminoglycan.

EXT gene can be altered in a variety of ways, including for example, by administering to cells a small molecule which interferes with EXT gene expression, by delivering to the cells a vector which directs the expression of an antisense or ribozyme molecule which inhibits expression of EXT genes, or by delivering a vector to said cells which directs the expression of an EXT gene. In the context of vectors, a wide variety of viral and non-viral (e.g. plasmid) vectors can be utilized, including for example, herpes viruses, retroviruses, adenoviruses, parvoviruses, alphaviruses, and pox viruses.

Within various embodiments, the glycosaminoglycan is heparan sulfate. Within other embodiments, the EXT gene is selected from the group consisting of EXT1 and EXT2. Within further embodiments, the expression of glycosaminoglycan can be increased or decreased, or the species (or ratio of species) of glycoaminoglycan altered (e.g., from chondroitin to heparan sulfate, or vice versa). Within these embodiments, cells may be either in vivo or ex vivo.

The methods as described above have a variety of applications. For example, within one aspect methods are provided for treating tumors, comprising the step of administering to a patient a gene delivery vehicle or vector which directs the expression of an EXT gene, such that growth of the tumor is diminished. Within a related aspect, methods are provided for treating cancers, comprising the step of administering to a patient a gene delivery vehicle or vector which directs the expression of an EXT gene, such that said cancer is treated. A wide variety of vectors may be utilized within the context of the present invention, including for example, “naked DNA” vectors, and viral vectors (e.g., delivery vehicles based upon retroviruses, herpes viruses, alphaviruses, adenoviruses and adeno-associated viruses). Within certain embodiments, the vehicle or vector may be directly administered into the tumor. In addition to gene delivery vehicles or vectors which direct the expression of an EXT gene, the vector may also direct the expression of an additional tumor suppressor gene (e.g., p53, BRCA1, or, Rb), and/or an immunomodulatory cofactor.

Also provided by the present invention are gene delivery vehicles or vectors which direct the expression of a nucleic acid molecule which inhibits the expression of an EXT gene. Within certain embodiments, the nucleic acid molecule is an antisense molecule or a ribozyme molecule. Also provided are cells (both in vivo and ex vivo) which contain a gene delivery vehicle as described herein. The cells may be of mammalian (e.g., human, rat, mouse etc.) or non-mammalian (e.g., C. elegans) origin.

Also provided by the present invention are assays for determining the ability of a compound to alter EXT activity. Within one aspect, such methods comprise the general step of introducing a compound into cells which express EXT, and determining whether the compound alters the quantity or species of glycosaminoglycan produced by the cell. A wide variety of methods may be utilized to assess the altered ability of a cell to express glycosaminoglycans, including for example, ELISA assays, assessing the cell for herpes infectivity, assaying the cells for alteration in the ability of glycosaminoglycan binding ligands to bind to the cell, or, assaying for RNA encoding EXT. In the context of such assays, the cell may be either a whole cell (e.g., sog 9 cells), or a cell membrane.

Also provided by the present invention are methods for identifying genes which have EXT-like activity, comprising the general step of expressing a nucleic acid molecule from a cell, and determining whether said cell exhibits an increased level of glycosaminoglycans. As noted above, cells a wide variety of methods may be utilized to assess the altered ability of a cell to express glycosaminoglycans, including for example, ELISA assays, assessing the cell for herpes infectivity, or assaying for RNA encoding an EXT.

In addition, the present invention provides transgenic non-human organisms such as mice or rats whose somatic and germ line cells contain a transgene encoding an EXT gene. Alternatively, “knockout” transgenic non-human organisms having a phenotype characterized by cancer or tumor formation, the phenotype being conferred by a transgene contained in the somatic and germ cells of the mouse, the transgene comprising a mutant EXT gene.

These and other aspects of the present invention will become evident upon reference to the following detailed description and attached drawings. In addition, various references are set forth herein which describe in more detail certain procedures or compositions (e.g., plasmids, etc.), and are therefore incorporated by reference in their entirety.

### BRIEF DESCRIPTION OF THE DRAWINGS

FIGS. 1A, 1B, 1C and 1D show that Sog9 cells transfected with EXT1 are susceptible to HSV-1 infection. Briefly, subconfluent cell monolayers were transfected with a control plasmid (a), or EXT1 (b) and after 30 h infected with R8102, a β-galactosidase expressing HSV-1 strain F, at 0.1 plaque forming units per cell. At 10 h post infection, cells were stained with X-gal. (c) Clonal sog9-EXT1 cells exhibit wild type levels of susceptibility to HSV-1 infection. Cell lines were infected with wild type HSV type 1 strain KOS and plaques were visualized after 3 days by staining with a 70% methanol/5% methylene blue solution. Values are averages of 4 determinations. The data represents the relative percent infectivity compared to an L cell control (100%). d, EXT1 restores HSV-1 attachment to sog9 cells. Cell monolayers were incubated with radiolabeled HSV-1 type 1 strain KOS on ice for 2 hours. The radioactivity associated with the monolayer was determined by liquid scintillation spectroscopy. Values are averages of 4 determinations. Open squares, mouse L cells; open circles, sog9 cells; closed circles, sog9-EXT1 cells.

FIGS. 2A-2C are a series of graphs which show that EXT1 expressing sog9 cells synthesize a heparan sulfate-like glycosaminoglycan. Briefly, monolayers of parental L (a), sog9 (b), and sog9-EXT1 (c) cells were grown for 3 days in the presence of [³⁵S]sulfate and D-[6-³H]glucosamine. Glycosaminoglycans were isolated and fractionated by HPLC. HS, elution position of heparan sulfate; CS, elution position of chondroitin sulfate. Open squares, D-[6-³H]glucosamine; closed diamonds, [³⁵S]sulfate. Note the change in scale between panel a and panels b,c.

FIGS. 3A and 3B are photographs which show that EXT1 is localized to the ER. Monolayers of CHO cells were transfected with either EXT1myc (a) or ΔNTMEXT1myc (b), and processed for indirect immunofluorescence using an anti-myc monoclonal antibody. Images were captured by confocal microscopy. The values of the prestained protein molecular weight markers are indicated in kilodaltons.

FIG. 4 is a Western blot which shows that EXT1myc is modified by high-mannose N-linked oligosaccharides. CHO cells were transfected with myc tagged EXT1 constructs for 30 hours. CHO cell lysates were digested or mock-digested with 1U endoH and subjected to polyacrylamide gel electrophoresis. Western blots were incubated with an anti-myc antibody and detected by chemiluminescence.

FIG. 5 is a series of schematic illustrations which shows the construction and activity of a variety of EXT1 constructs. Constructs were tested for activity by transfection into sog9 cells prior to infection with HSV-1. Subcellular localization of constructs was tested by indirect immunofluorescence analysis using a monoclonal antibody directed against the myc-epitope tag. (−) no activity above background, (+) activity equal to wild-type EXT1.

FIGS. 6A and 6B depict the nucleic acid sequence of EXT1. FIGS. 6C and 6D depict the amino acid sequence of EXT1.

FIGS. 7A and 7B depict the nucleic acid sequence of EXT2. FIGS. 7C and 7D depict the amino acid sequence of EXT2.

FIG. 8 is a schematic illustration of a representative EXT gene.

### DETAILED DESCRIPTION OF THE INVENTION

### DEFINITIONS

Prior to setting forth the invention, it may be helpful to an understanding thereof to first set forth definitions of certain terms that will be used hereinafter.

“Gene delivery vehicle”, or “vector” when utilized in the context of the present invention refers to constructs which are capable of expressing one or more gene(s) or sequence(s) of interest in a host cell. Representative examples of such vehicles include nucleic acid expression vectors, naked DNA (which may, within certain embodiments, be encased within a lipid membrane), viral vectors (e.g., alphaviruses, adenoviruses, adeno-associated viruses, herpes viruses and retroviruses) and certain eukaryotic cells (e.g., producer cells).

As noted above, the present invention provides methods for altering glycosaminoglycan expression in cells (i.e., either up-regulation or down-regulation). Such general methods have a variety of applications which are all based upon the surprising and unexpected discovery that EXT genes have a profound effect on cell surface glycosaminoglycans. Understanding the role of EXT genes in expression of cell surface glycosaminoglycans has important application not only in understanding viral binding to cells, but in the treatment of a wide variety of diseases. For example, within one aspect methods are provided for treating tumors, comprising the step of administering to a patient a gene delivery vehicle or vector which directs the expression of an EXT gene, such that growth of the tumor is diminished.

In order to further and understanding of the inventions described herein, set forth below is a brief discussion of EXT sequences, gene delivery vehicles which may be utilized within the context of the present invention, other nucleic acid molecules or sequences of interest which may be expressed by the gene delivery vehicles described herein, and other assays and methods which utilize the discovery described above.

### EXT SEQUENCES

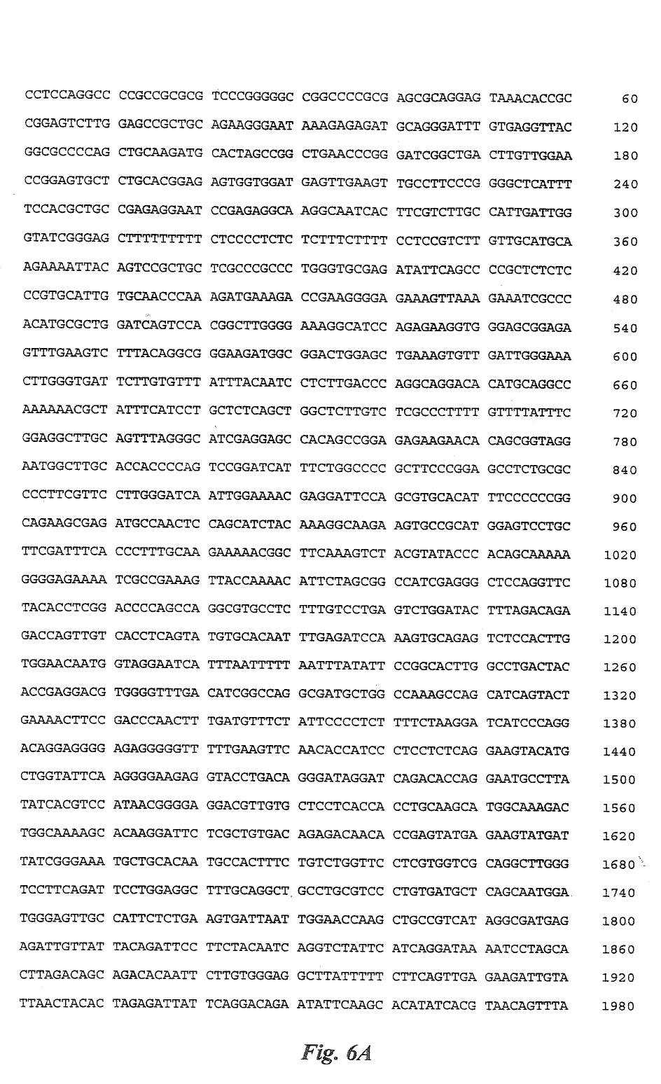

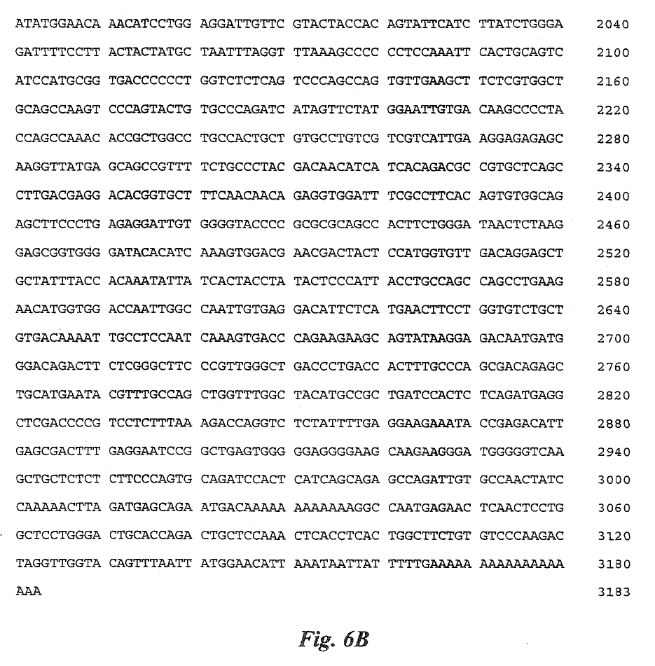

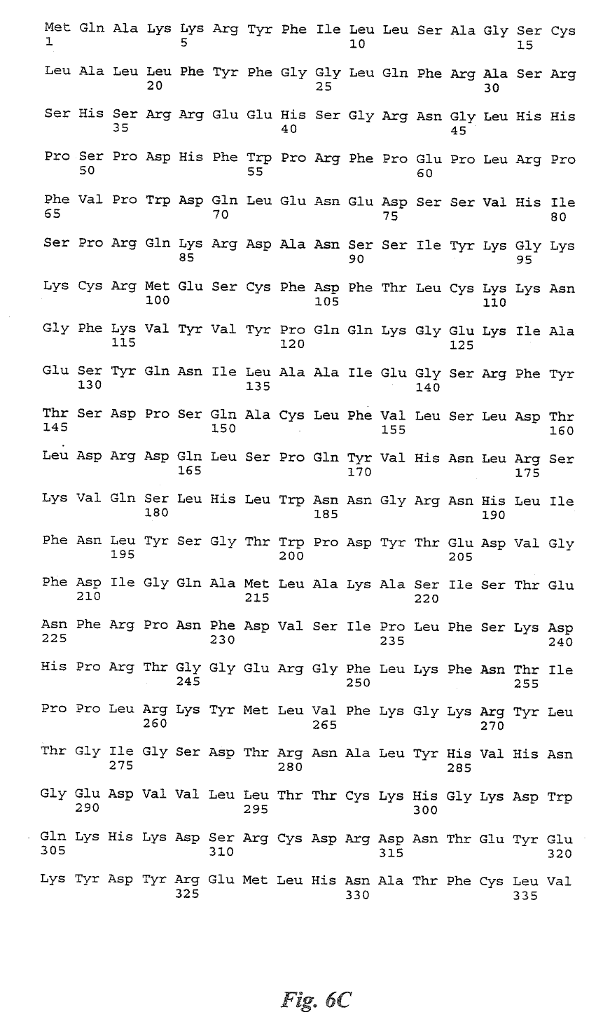

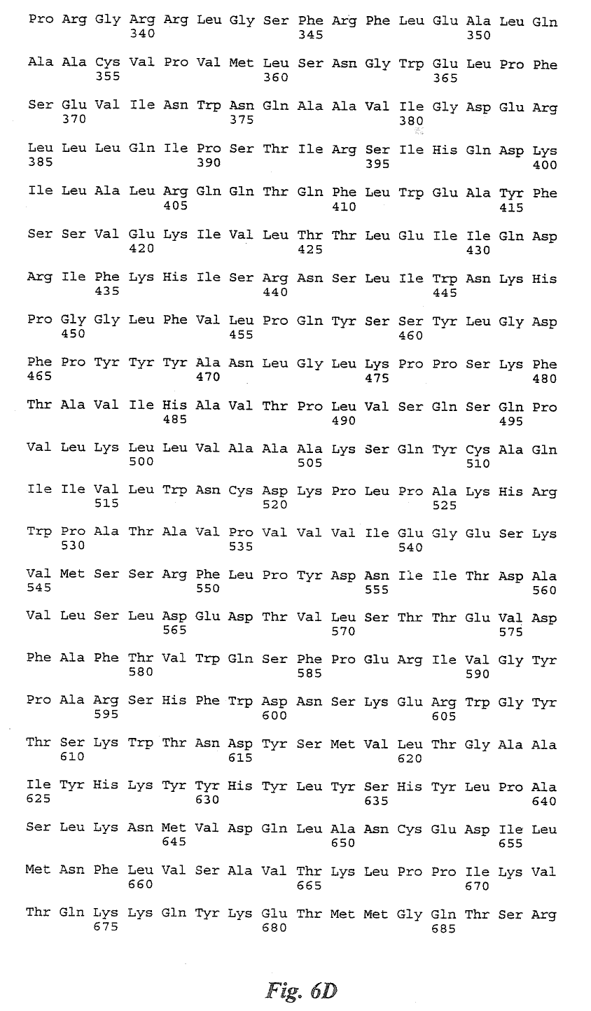

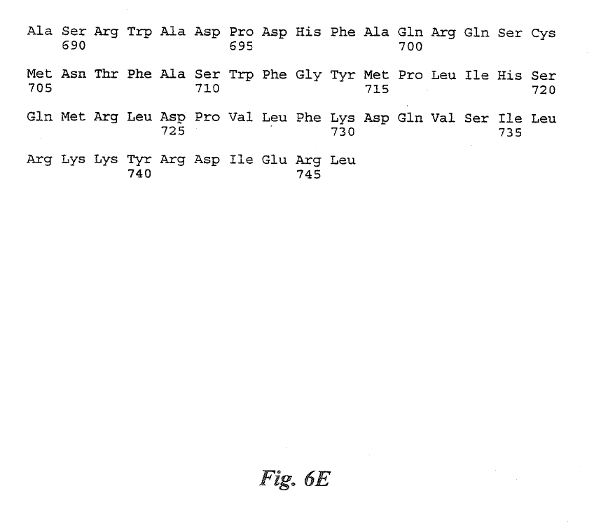

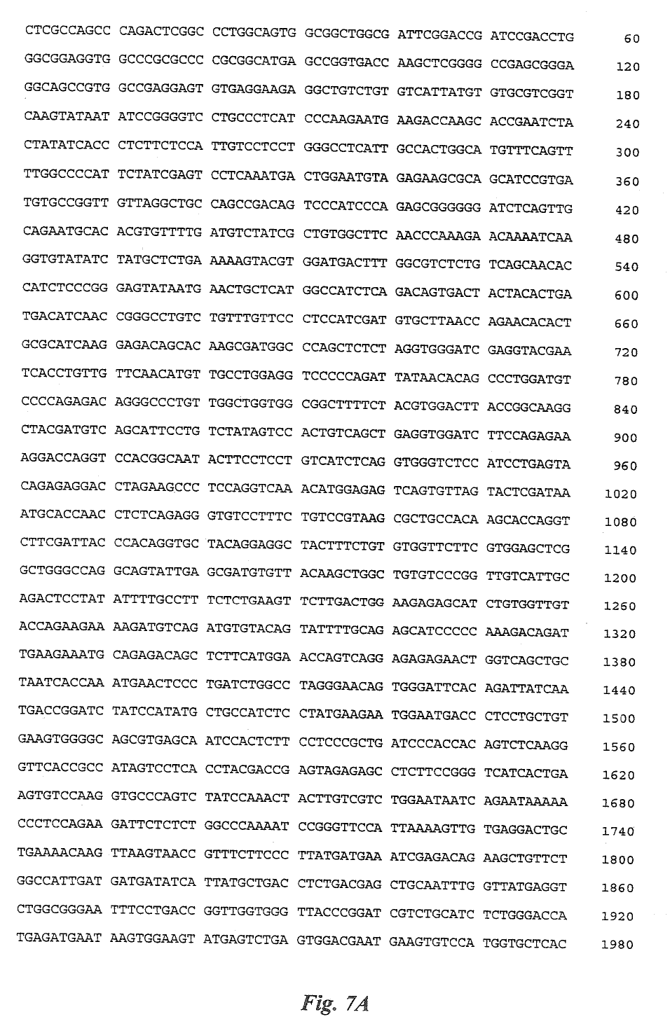

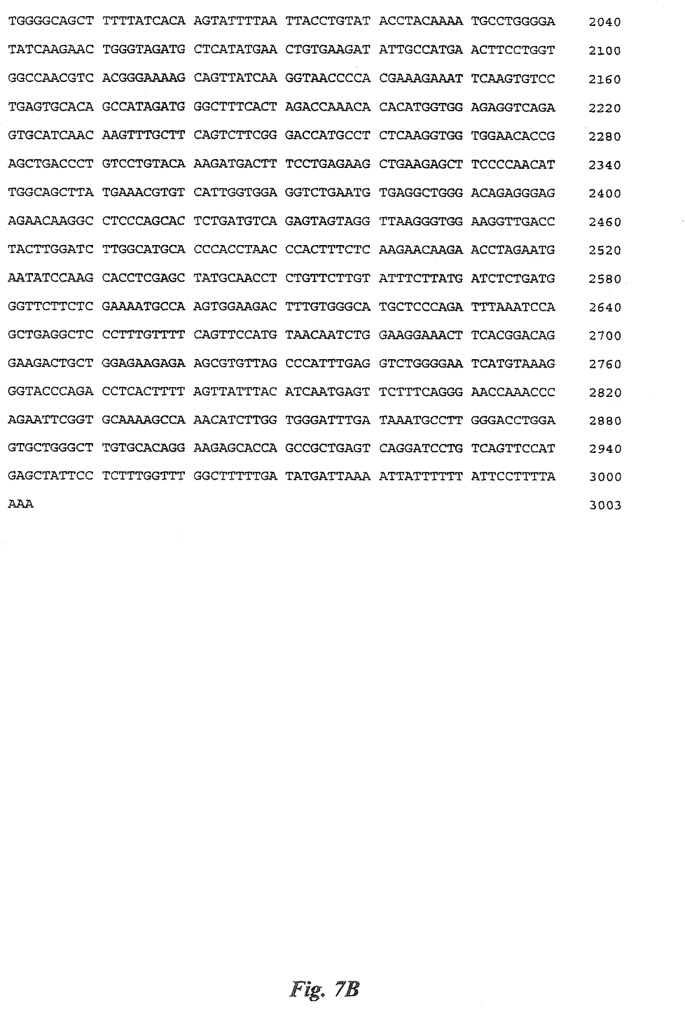

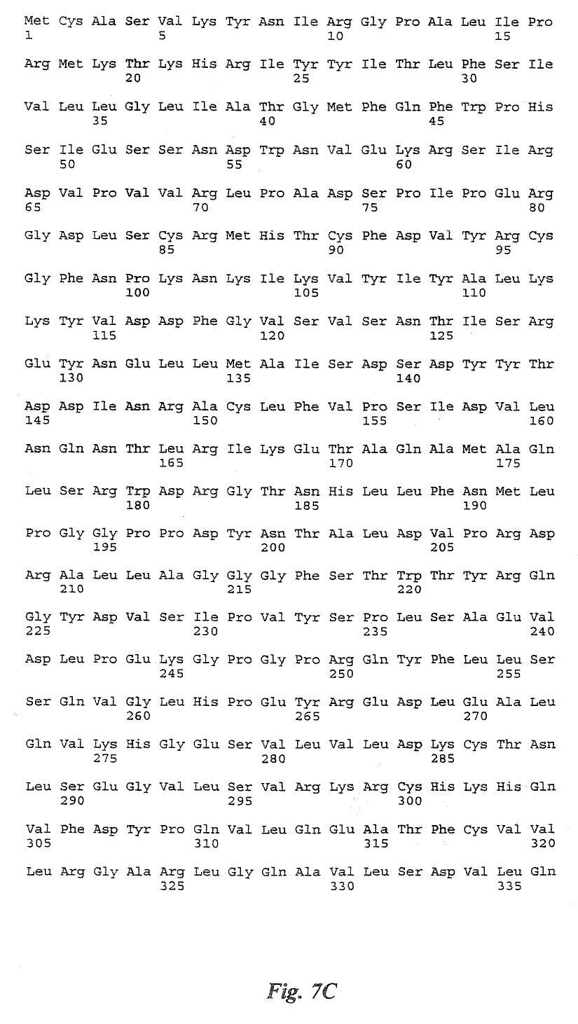

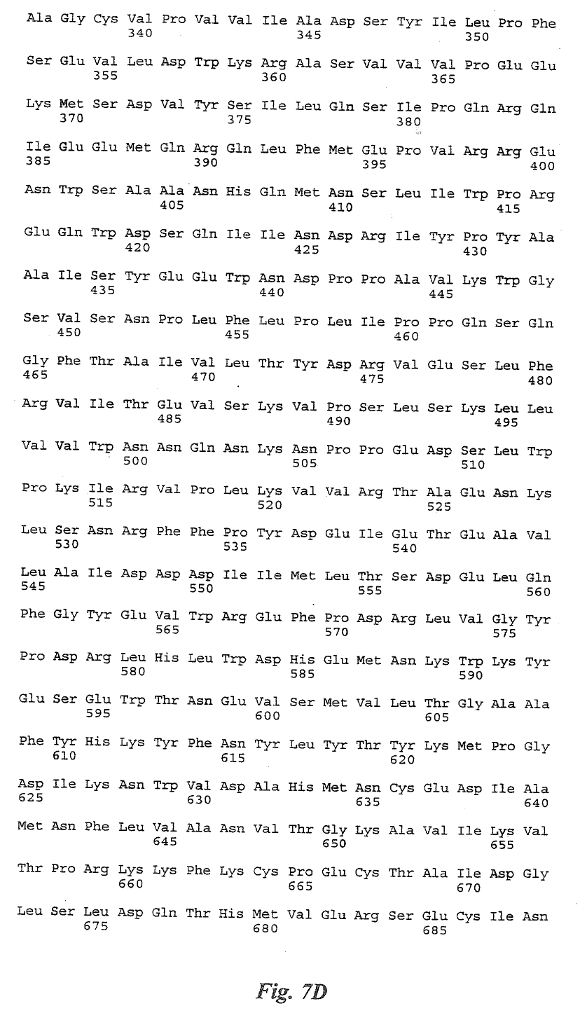

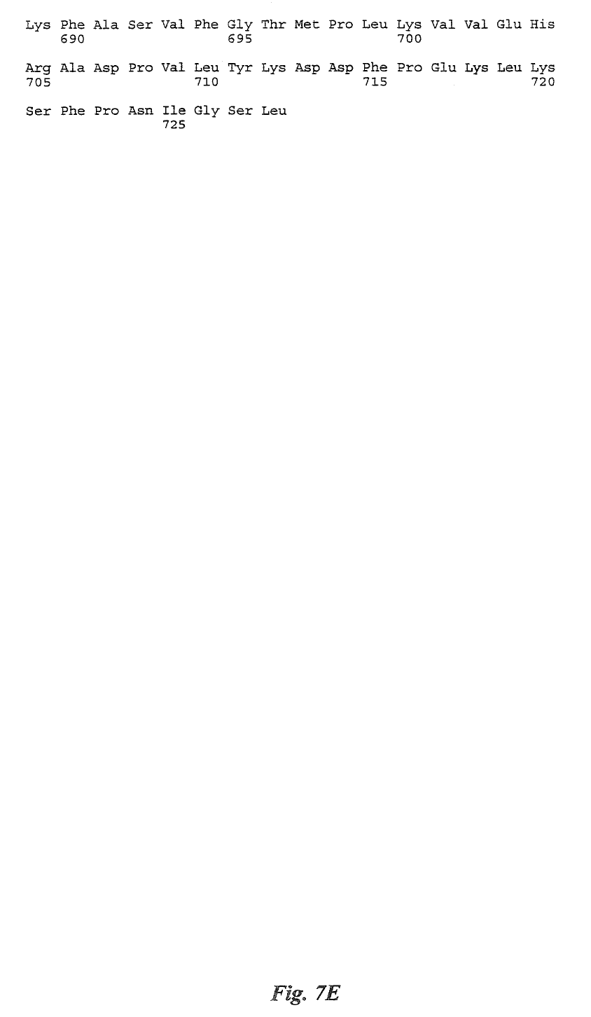

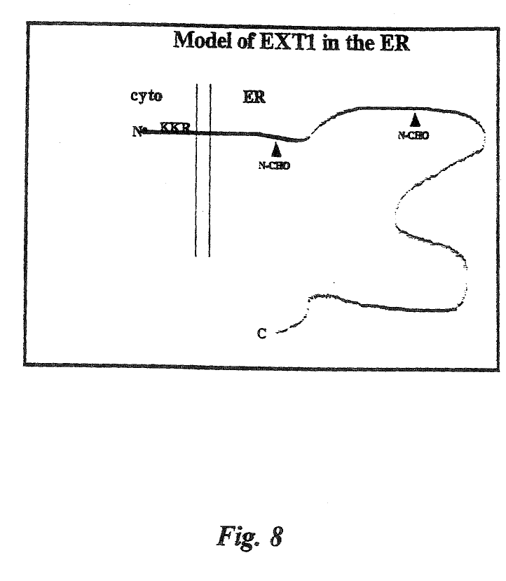

EXT genes have been linked to hereditary multiple exostoses, although the exact nature of the linkage and the direct association with the disease has been, to date, unknown. Genes which encode EXT have been deposited with Genbank: e.g., EXT 1 (Genbank Accession No. S79639) and EXT2 (Genbank Accession No. U72263). The DNA sequence and encoded protein of EXT 1 and EXT2 are also provided in FIGS. 6 and 7. Briefly, EXT1 is predicted to encode a 746 amino acid protein from a 3.4 kb mRNA that has been shown to be expressed in a large number of tissues, with the highest expression in the liver (Ahn et al., Nature Genetics 11:137-43, 1995). The promoter region of EXT1 contains GC and CAAT boxes, but no TATA box, suggesting a form of regulation characteristic of housekeeping genes. This is consistent with the ubiquitous expression of EXT1 (Ludecke et al., Genomics 40:351-4, 1997).

EXT2 is somewhat similar to EXT1 in terms of its sequence (see Clines et al., “The Structure of the Human Multiple Exostoses 2 Gene and Characterization of Homologs in Mouse and Caenorhabditis elgans” Genome Res. 7:359-367, 1997). The expression pattern of EXT2 is also somewhat similar to EXT1, although several minor transcripts are also expressed, suggesting that alternatively spliced forms of the RNA are synthesized that could have biological relevance in the disease (Stickens et al., Nature Genetics 14:25-32, 1996.

EXTL is a recently cloned member of the same family (see Wise et al., “Identification and Localization of the Gene for EXTL, a Third Member of the Multiple Exostoses Gene Family”, Genome Res. 7:10-16, 1997. The sequence of EXTL is deposited under GenBank accession no. U67191. The biological activity of EXTL has not been reported.

Although several EXT genes have been disclosed in the Sequence Listing and Figures provided herein, it should be understood that within the context of the present invention, reference to one or more of these genes includes related genes that are substantially similar to the genes (and, where appropriate, the polypeptide that are encoded by the genes and their derivatives) described herein. As used herein, a nucleotide sequence is deemed to be “substantially similar” if: (a) the nucleotide sequence is derived from the coding region of the described genes and includes, for example, portions of the sequence or allelic variations of the sequences discussed above, or alternatively, encodes an EXT-like activity (e.g., the ability to alter the level or species of glycosaminoglycan on cells); (b) the nucleotide sequence is capable of hybridization to nucleotide sequences of the present invention under conditions of moderate, or, high stringency (see Sambrook et al., Molecular Cloning: A Laboratory Manual, 2nd ed., Cold Spring Harbor Laboratory Press, NY, 1989); (c) the protein encoded by the nucleotide sequence has at least 22%, 30%, or 40% identity, or, 49% or greater similarity to the protein encoded by any of the EXT genes disclosed herein, or, (d) the DNA sequences are degenerate as a result of the genetic code to the DNA sequences defined in (a), (b), or (c). Further, the nucleic acid molecule disclosed herein includes both complementary and non-complementary sequences, provided the sequences otherwise meet the criteria set forth herein. Within the context of the present invention, high stringency means standard hybridization conditions (e.g., 5XSSPE, 0.5% SDS at 65° C., or the equivalent) while very high stringency means conditions of hybridization such that the nucleotide sequence is able to selectively hybridize to a single allele of an EXT gene.

The EXT gene may be isolated from genomic DNA or cDNA. Genomic DNA libraries can be constructed in vectors such as BACs (bacterial artificial chromosomes), YACs (yeast artificial chromosomes), bacteriophage vectors such as λ EMBL3, λgt10, cosmids or plasmids. cDNA libraries can also be constructed in bacteriophage vectors, plasmids, or other vectors which are suitable for screening. Such libraries may be constructed using methods and techniques known in the art (see Sambrook et al., Molecular Cloning: A Laboratory Manual, Cold Spring Harbor Press, 1989) or purchased from commercial sources (e.g., Clontech). Within one embodiment, an EXT gene can be isolated by PCR performed on genomic DNA, cDNA or DNA in libraries, or by probe hybridization of genomic DNA or cDNA libraries. Primers for PCR and probes for hybridization screening may be designed based on the DNA sequence of the EXT genes presented herein (or see Wise et al., supra, or, Clines et al., supra). Briefly, the primers should preferably not have self-complementary sequences nor have complementary sequences at their 3′ end (to prevent primer-dimer formation). In addition, the primers should have a GC content of about 50% and contain restriction sites. The primers are annealed to cDNA and sufficient cycles of PCR are performed to yield a product readily visualized by gel electrophoresis and staining. The amplified fragment is purified and inserted into a vector, such as λgt10 or pBS(M13+), and propagated.

An oligonucleotide hybridization probe suitable for screening genomic or cDNA libraries may be designed based on the sequence provided herein. Preferably, the oligonucleotide is 20-30 bases long. Such an oligonucleotide may be synthesized by automated synthesis. The oligonucleotide may be conveniently labeled at the 5′ end with a reporter molecule, such as a radionuclide, (e.g., ³²P) or biotin. The library is plated as colonies or phage, depending upon the vector, and the recombinant DNA is transferred to nylon or nitrocellulose membranes. Following denaturation, neutralization, and fixation of the DNA to the membrane, the membranes are hybridized with the labeled probe. The membranes are washed and the reporter molecule detected. The hybridizing colonies or phage are isolated and propagated. Candidate clones or PCR amplified fragments may be verified as containing EXT DNA by any of various means. For example, the candidate clones may be hybridized with a second, nonoverlapping probe or subjected to DNA sequence analysis. In these ways, clones containing an EXT gene, which are suitable for use in the present invention can be isolated.

Assays which may be utilized to identify clones containing genes or other sequences having EXT activity are described in more detail below. As an example, one such method comprises the general step of expressing a nucleic acid molecule from a cell, and determining whether the cell exhibits an increased level of glycosaminoglycans. As noted above, cells a wide variety of methods may be utilized to assess the altered ability of a cell to express glycosaminoglycans, including for example, ELISA assays, assessing the cell for herpes infectivity, or assaying for RNA encoding EXT.

### GENE DELIVERY VEHICLES

As noted the present invention provides a wide variety of gene delivery vehicles (also referred to as vectors or constructs) which can be utilized to delivery the genes described herein to a host cell or organism. Representative examples of such constructs include a variety of non-viral and viral vectors, as described below, as well as cells which are capable of producing such vectors.

For example, within one aspect of the invention retroviral vectors may be utilized as gene delivery constructs suitable for delivering the above-noted expression constructs. Representative examples of such retroviral vectors include those described within EP 0,415,731, WO 90/07936, WO 91/0285, WO 94/03622, WO 93/25698, WO 93/25234, U.S. Pat. No. 5,219,740, WO 93/11230, WO 93/10218, U.S. Pat. No. 4,777,127, EP 0,345,242 and WO 91/02805).

Other examples of suitable gene delivery constructs may be found in the Herpesvirus family. Briefly, suitable members of the Herpesviridae include both primate Herpesviruses, and nonprimate Herpesviruses such as avian Herpesviruses. Representative examples of suitable Herpesviruses include Herpes Simplex Virus Type 1 (McKnight et al., Nuc. Acids Res. 8:5949-5964, 1980; Fields et al., Fundamental Virology, Raven Press, N.Y. (1986)), Herpes Simplex Virus Type 2 (Swain and Galloway, J. Virol. 46:1045-1050, 1983), Varicella Zoster Virus (Davison and Scott, J. Gen. Virol. 67:1759-1816, 1986) and Epstein-Barr virus (Baer et al., Nature (London) 310:207-311, 1984).

Herpesviruses may be readily obtained from commercial sources such as the American Type Culture Collection (“ATCC”, Rockville, Md.). Deposits of certain of the above-identified Herpesviruses may be readily obtained from the ATCC, for example: ATCC No. VR-539 (Herpes simplex type 1); ATCC Nos. VR-734 and VR-540 (Herpes Simplex type 2); and ATCC No. VR-586 (Varicella Zoster Virus). Herpesviruses may also be readily isolated and identified from naturally occurring sources (e.g., from an infected animal).

In addition to retroviral vectors and Herpes viral vectors, a wide variety of other gene delivery constructs may be utilized to deliver expression cassettes, including for example constructs derived from adenovirus (Rosenfeld et al., Science 252:431-434, 1991; Kolls et al., PNAS 91(1):215-219, 1994; Kass-Eisler et al., PNAS 90(24):11498-502, 1993; Levrero et al., Gene 101(2):195-202, 1991); and Guzman et al., Circulation 88(6):2838-48, 1993; alphaviruses such as Semliki Forest Virus and Sindbis Virus (Xiong et al., Science 243:1188, 1989; Raju and Huang, J. Vir. 65(5):2501-2510, 1991; Hertz and Huang, J. Vir. 66(2):857-864, 1992, WO 92/10578; WO 95/07994; U.S. Pat. No. 5,091,309); influenza virus (Luytjes et al., Cell 59:1107-1113, 1989; McMicheal et al., N. Eng. J. Med. 309:13-17, 1983; and Yap et al., Nature 273:238-239, 1978); pox viruses, such as canary pox virus or vaccinia virus (Fisher-Hoch et al., PNAS 86:317-321, 1989; Flexner et al., Ann. N.Y. Acad. Sci. 569:86-103, 1989; U.S. Pat. Nos. 4,603,112, 4,769,330 and 5,017,487; WO 89/01973); SV40 (Mulligan et al., Nature 277:108-114, 1979); parvovirus such as adeno-associated virus (Samulski et al., J. Vir. 63:3822-3828, 1989; Plotte et al., G. Biol. Chem. 268:3781-3790, 1993; Flotte et al., PNAS 90(22):10613-10617, 1993; WO 95/13365); and HIV (Poznansky, J. Virol. 65:532-536, 1991).

Other non viral vector systems that may also be utilized include a variety of nucleic acid based transcription systems (e.g., based on T7 or SP6 promoters, see generally, WO 95/07994). Such vector systems may be administered and prepared as described above (e.g., in liposomes, condensed with polycations, or linked to a ligand).

### SEQUENCES OF INTEREST

In addition to delivering an EXT gene to a cell or organism of interest, the gene delivery vehicles of the present invention may also deliver other nucleic acid molecules which may have beneficial effects on the treatment, diminution, or prevention of cancer or tumors. Representative examples of other suitable nucleic acid molecules include a wide variety of tumor suppressor genes, and immunomodulatory cofactors.

A. Tumor Suppressor Genes

A wide variety of tumor supressor genes may be delivered by the gene delivery vehicles of the present invention. Including for example, p53, RB, and the like. Briefly, many tumor types display alterations in GAGs (Tuszynski et al., Acta Haematologica 97:29-39, 1997; Schamhart and Kurth, Urological Research 25:S89-96, 1997; Esko et al., Science 241:1092-6, 1988; Iida et al., Seminars in Cancer Biology 7:155-62, 1996; Iozzo and Cohen, Exs 70:199-214, 1994). Some poorly differentiated lung tumors have markedly altered patterns of HS proteoglycan expression, which may contribute to their invasive phenotype (Nackaerts et al., International Journal of Cancer 74:335-45, 1997). In HME, changes in the structure of cell surface proteoglycans may interfere with the signal transduction systems that govern cellular growth and differentiation, resulting in tumor formation. Some proteins involved in cell adhesion and cell signaling such as APC (adenomatous polyposis coli), DCC (deleted in colon carcinoma), and NF2 (neurofibromatosis type 2) are tumor suppressor genes (Rubinfeld et al., Science 262:1731-4, 1993; Fearon and Vogelstein, Cell 61:759-67, 1990; Trofatter et al., Cell 75:826, 1993). Utilizing the compositions and methods of the present invention, gene delivery vehicles may be designed to co-express (e.g., utilizing an IRES or separate promoters) a tumor suppressor gene in addition to an EXT gene.

For example, p53, is a tumor suppressor gene which negatively regulates the cell cycle (see, eg., Mercer, et al., “Negative Growth Regulation in a Glioblastoma Tumor Cell Line that Conditionally Expresses Human Wild-Type p53,” Proc. Natl. Acad. Sci. USA 87:6166-6170, 1990; Diller et al., “p53 Functions as a Cell Cycle Control Protein in Osteosarcomas,” Molecular and Cellular Biology 10(11):5772-5781, 1990; Baker et al., “Suppression of Human Colorectal Carcinoma Cell Growth by Wild-Type p53,” Science 249:912-915 (1990); Harlow et al., “Molecular Cloning and In Vitro Expression of a cDNA Clone for Human Cellular Tumor Antigen p53.” Molecular and Cellular Biol. 5(7):1601-1610, 1985). Forms of cancer which are due to, or associated with p53 mutations can now be treated, at least in part, utilizing gene therapy techniques (see U.S. Pat. Nos. 5,543,220, 5,011,773 and 5,104,571).

Retinoblastoma is a childhood eye cancer associated with the loss of a gene locus designated Rb, which is located in chromosome band 13q14. A gene from this region has been cloned which produces a nuclear phosphoprotein of about 110 kd (Friend et al., Nature 323:643, 1986; Lee et al., Science 235:1394, 1987; and Fung et al., Science 236:1657, 1987). Rb is believed to be a negative regulator of cellular proliferation, and has a role in transcriptional control and cell-cycle regulation (see, Huang et al., “Suppression of the Neoplastic Phenotype by Replacement of the RB Gene in Human Cancer Cells,” Science 242:1563-1566, 1988; Huang et al., “A cellular protein that competes with SV40 T antigen for binding to the retinoblastoma gene product,” Nature 350:160-162, 1991; Huang et al., “Two distinct and frequently mutated regions of retinoblastoma protein are required for binding to SV40 T antigen,” The EMBO Journal 9(6):1815-1822, 1990; Lee et al., “Molecular mechanism of retinoblastoma gene inactivation in retinoblastoma cell line Y79,” Proc. Natl. Acad. Sci. USA 85:6017-6021, 1988. Lee et al., “Human Retinoblastoma Susceptibility Gene: Cloning, Identification, and Sequence,” Science 235:1394-1399, 1987; Shew et al., “C-terminal truncation of the retinoblastoma gene product leads to functional inactivation,” Proc. Natl. Acad. Sci. USA 87:6-10, 1990; Shohat et al., “Inhibition of cell growth mediated by plasmids encoding p53 anti-sense,” Oncogene 1:277-283, 1987. Smith et al., “Expression of the p53 oncogene in acute myeloblastic leukemia,” J. Exp. Med. 164:751-761, 1986.

Other tumor suppressor genes include BRCA1 and BRAC2, VHL, WT1, nm23. multiple endocrine neoplasia type 1 (MEN1) which is involved in parathyroid adenomas. (Cryns VL, et al., Genes Chromosomes Cancer 13 (1) 9-17, (1995); DCC, a tumor suppressor gene deleted in colorectal cancer. (Kolodziej PA, Curr Opin Genet Dev 7 (1) 87-92, 1997); p15INK4B and p16INK4 genes which encode cell cycle regulating proteins. (Heyman M; et al., Lymphoma 23 (3-4) 235-45, (1996); A CDKI, p16, and its gene, CDKN2 (MTS1/p16INK4A), identified on chromosome 9p21. Alterations frequently occur in the CDKN2 tumor suppressor gene. (Uchida T; et al. Lymphoma 24 (5-6) 449-61, (1997); p16 tumor suppressor gene , a new molecular event in pancreas cancer. Mutation of cyclin-dependent kinases also may be involved in pancreas carcinogenesis. Loss or mutation of a new candidate tumor suppressor, DPC4 (deleted in pancreas carcinoma locus 4), is reported in pancreas cancer. (Chu TM J Clin Lab Anal 11 (4) 225-31, (1997); CDKN2, p53, and E-cadherin, other cell adhesion genes, are tumour suppressor genes, inactivated in many tumour types, including the late stages of prostate cancer; (MacGrogan D; et al. Cancer Biol 8 (1) 11-19, (1997); p16, p15, VHL, and E-cadherin hypermethylation correlates with transcriptional repression. (Baylin SB; et al. Adv Cancer Res 72 141-96 (1998); CDKN2 (9p21), RB1 (13q14), and TP53 (17p13), recurrent deletions encompass sites of tumor suppressor genes commonly inactivated in lung carcinomas. (Testa JR; et al. Cancer Genet Cytogenet 95 (1) 20-32, (1997); Inactivating mutations of the tumour suppressor genes TP53, CDKN2 and SMAD4, genetic defects of pancreatic cancer. (Lemoine NR Digestion 58 (6) 550-6, (1997); DPC4 and MADR2 of the transforming growth factor beta (TGF-beta) pathway, related to colorectal cancer, tumor-suppressor genes (Gryfe R; et al. Curr Probl Cancer 21 (5) 233-300 (1997); Tumor-suppressor genes, p16INK4a and NF2,. change in mesothelioma. (Lechner JF; et al. Environ Health Perspect 105 Suppl 5, 1061-67 (1997); Inactivation of multiple tumor suppressor genes, p53, DPC4, p16 and BRCA2, associated with pancreatic cancer. Hruban RH, et al. Surg Oncol Clin N Am 7 (1) 1-23 (1998); Inactivation of multiple tumor suppressor genes, p53, DCC, APC, MCC, BRCA1, and WAF1/CIP1, associated with pancreatic cancer. Gao X, et al. Adv Exp Med Biol 407 41-53 (1997);

B. Immunomodulatory Cofactors

Immunologically active molecules (Immunomodulatory Cofactors) may also be expressed by the expression cassettes and gene delivery constructs described herein. As utilized within the context of the present invention, it should be understood that “immunologically active molecules” refers to those molecules which can either increase or decrease the recognition, presentation or activation of a cell-mediated or humoral immune response. Representative examples of immunologically active molecules include lymphokines such as IL-1, IL-2, IL-3, IL4, IL-5, IL-6, IL-7, IL-8, IL-9, IL-10, IL-11, IL-12 (WO 90/05147; EPO 433,827), IL-13 (WO 94/04680), IL-14, IL-15, α, β,or γ-interferon, GM-CSF, M-CSF-1, G-CSF, ICAM-1 (Simmons et al., Nature 331:624-627, 1988), ICAM-2 (Singer, Science 255:1671, 1992), β-microglobulin (Parnes et al., PNAS 78:2253-2257, 1981), HLA Class I, HLA Class II molecules, B7 (Freeman et al., J. Immun. 143:2714, 1989), and B7-2, as well as their respective receptors. Other biologically active molecules that may likewise be utilized in the context of the present invention include neurotrophins such as nerve growth factor (NGF), brain derived growth neurotrophic factor (BDGF), glial-derived neurotrophic factor (GDNF), neurotrophin-3 (NT-3), tumor necrosis factor (TNF) as well as their respective receptors.

As noted above within certain aspects of the present invention, the expression cassettes and gene delivery constructs described herein may direct the expression of more than one heterologous sequence. Such multiple sequences may be controlled either by a single promoter, or alternatively, by additional secondary promoters (e.g., Internal Ribosome Entry Sites or “IRES”). Within further embodiments of the invention, gene delivery constructs are provided which direct the expression of heterologous sequences which act synergistically (e.g., a disease-associated antigen, and an immunologically active molecule, such as IL-2, IL-12 or β-interferon).

C. Antisense and Ribozyme Sequences

The gene delivery vehicles of the present invention may also be constructed so as to deliver an antisense or ribozyme sequence. Representative examples of antisense and ribozyme sequences include those sequences which inhibit, for example, tumor cell growth, by preventing the cellular synthesis of critical proteins. Examples of such antisense sequences include antisense ABL (Fainstein et al., Oncogene 4:1477-1481, 1989), antisense HER2 (Coussens et al., Science 230:1132-1139, 1985), antisense myc (Stanton et al., Nature 310:423-425, 1984),and antisense ras.

Representative examples of ribozyme sequences include hammerhead ribozymes (for example, as described by Forster and Symons, Cell 48:211-220, 1987; Haseloff and Gerlach, Nature 328:596-600, 1988; Walbot and Bruening, Nature 334:196, 1988; Haseloff and Gerlach, Nature 334:585, 1988) and hairpin ribozymes (for example, as described by Haseloff et al., U.S. Pat. No.5,254,678 and Hempel et al., European Patent Publication No. 0 360 257) which have the ability to specifically target, cleave and inactivate RNA or mRNA. Briefly, the sequence requirement for the hairpin ribozyme is any RNA sequence consisting of NNNBN*GUCNNNNNNNN (where N*G is the cleavage site, wherein B is any of G, C, or U, and where N is any of G, U, C, or A) (Sequence I.D. No. 5). The sequence requirement at the cleavage site for the hammerhead ribozyme is any RNA sequence consisting of NUX (where N is any of G, U, C, or A and X represents C, U or A) can be targeted. Accordingly, the same target within the hairpin leader sequence, GUC, is useful for the hammerhead ribozyme. The additional nucleotides of the hammerhead ribozyme or hairpin ribozyme is determined by the target flanking nucleotides and the hammerhead consensus sequence (see Ruffner et al., Biochemistry 29:10695-10702, 1990).

Sequences which encode the above-described heterologous genes may be readily obtained from a variety of sources. For example, plasmids which contain sequences that encode immunologically active molecules may be obtained from a depository such as the American Type Culture Collection (ATCC, Rockville, Md.), or from commercial sources such as British Bio-Technology Limited (Cowley, Oxford, England). Representative sources sequences which encode the above-noted immune accessory molecules include ATCC No. 20663 (which contains sequences encoding alpha interferon), ATCC Nos. 31902 and 39517 (which contains sequences encoding beta interferon), ATCC Nos. 39405, 39452, 39516, 39626 and 39673 (which contains sequences encoding Interleukin-2), ATCC Nos. 59399, 59398, and 67326 (which contain sequences encoding Interleukin-3), ATCC No. 57592 (which contains sequences encoding Interleukin-4), ATCC Nos. 59394 and 59395 (which contain sequences encoding Interleukin-5), and ATCC No. 67153 (which contains sequences encoding Interleukin-6). It will be evident to one of skill in the art that one may utilize either the entire sequence of the protein, or an appropriate portion thereof which encodes the biologically active portion of the protein.

Alternatively, known cDNA sequences which encode a gene of interest may be obtained from cells which express or contain such sequences. Briefly, within one embodiment mRNA from a cell which expresses the gene of interest is reverse transcribed with reverse transcriptase using oligo dT or random primers. The single stranded cDNA may then be amplified by PCR (see U.S. Pat. Nos. 4,683,202, 4,683,195 and 4,800,159. See also PCR Technology: Principles and Applications for DNA Amplification, Erlich (ed.), Stockton Press, 1989 all of which are incorporated by reference herein in their entirety) utilizing oligonucleotide primers complementary to sequences on either side of desired sequences. In particular, a double stranded DNA is denatured by heating in the presence of heat stable Taq polymerase, sequence specific DNA primers, ATP, CTP, GTP and TTP. Double-stranded DNA is produced when synthesis is complete. This cycle may be repeated many times, resulting in a factorial amplification of the desired DNA.

Sequences which encode the above-described genes may also be synthesized, for example, on an Applied Biosystems Inc. DNA synthesizer (e.g, ABI DNA synthesizer model 392 (Foster City, Calif.)).

### ASSAYS

The present also provides a variety of assays related to EXTs. For example, within one aspect of the invention assays are provided for determining the ability of a compound to alter EXT activity, comprising the general step of introducing a compound into cell which express EXT, and determining whether said compound alters the quantity or species of glycosaminoglycan produced by the cell

Within other aspects of the invention, methods are provided for determining EXT activity in cells, comprising the steps of (a) obtaining a nucleic acid molecule from a first cell which encodes EXT, (b) expressing the nucleic acid molecule in a second cell, and (c) determining whether expression of the nucleic acid molecule in said second cell alters glycosaminoglycan quantity of species of the first cell.

Within a related aspect, methods are provided for identifying genes which have EXT activity, comprising the general step of expressing a nucleic acid molecule from a cell, and determining whether the cell exhibits an altered level or species of glycosaminoglycans.

Finally, the present invention also provides methods for determining tumorigenicity of a cell, comprising the general step of assessing the cell of an altered or mutant form of an EXT gene that results in altered quantity or species of glycosaminoglycan production.

Representative assays for determining alteration in the quantity or species of glycosaminoglycan include ELISAs, and assaying cells for herpes infectivity (e.g., infection by a herpesvirus such Herpes Simplex Virus Type 1 (McKnight et al., Nuc. Acids Res. 8:5949-5964, 1980; Fields et al., Fundamental Virology, Raven Press, N.Y. (1986)), Herpes Simplex Virus Type 2 (Swain and Galloway, J. Virol. 46:1045-1050, 1983), Varicella Zoster Virus (Davison and Scott, J. Gen. Virol. 67:1759-1816, 1986) and Epstein-Barr virus (Baer et al., Nature (London) 310:207-311, 1984). Alteration in the quantity or species of glycosaminoglycan can also be determined by assessing the ability of glycosaminoglycan binding-ligands (such as, for example, a heparin-binding ligand) to bind to the cell.

One assay that may be utilized in order to assess alteration of glycosaminoglycan production by a cel is the glycosyltransferase assay generally described by Lindhold and Lindahl (see J. Biochem, 287:21-29, 1992).

As noted above, representative examples of glycosaminoglycans which may be detected or assayed for include heparin or heparan-sulfate, chondrotin or chondrotin sulfate, keratin sulfate, dermitan sulfate, and hyaluronic acid. As utilized herein, it should be understood that the term ‘cells’, at least for purposes of assay, includes both intact whole cells and cell membranes.

### ADDITIONAL METHODS AND COMPOSITIONS

In other aspects of the present invention, the gene delivery vehicles described herein are administered to a patient such as a warm-blooded animal (e.g., a human, monkey, cow, sheep, dog, cat, rat or mouse), or other-type of animal (e.g., fish) for the treatment, diminution, or prevention of a cancer or tumor. Such delivery vehicles may be introduced in vivo, using the techniques describe below, i.e., stereotactical microinjection, or ex vivo to cells which are then transplanted within the patient.

The present invention also provides for non-human animal models which are deficient in, or over express EXT genes. For example, gene delivery vehicles of the present invention may be utilized to generate non-human transgenic animals such as mice, rats, rabbits, sheep, dogs and pigs (see Hammer et al. (Nature 315:680-683, 1985), Palmiter et al. (Science 222:809-814, 1983), Brinster et al. (Proc. Natl. Acad. Sci. USA 82:4438-4442, 1985), Palmiter and Brinster (Cell 41:343-345, 1985) and U.S. Pat. No. 4,736,866). For example, within one embodiment an expression cassette may be introduced into pronuclei of fertilized eggs, for example, by microinjection. Integration of the injected DNA may be detected by blot analysis of DNA from tissue samples. It is preferred that the introduced DNA be incorporated into the germ line of the animal so that it is passed on to the animal's progeny. Such techniques allow for, within preferred embodiments, tissue-specific (e.g., neuronal cell) expression of a desired sequence of interest.

As utilized within the context of the present invention, the term “treatment” refers to reducing or alleviating symptoms in a subject, preventing symptoms from worsening or progressing, inhibition or elimination of the causative agent, or prevention of the infection or disorder in a subject who is free therefrom. Thus, for example, treatment of cancer or a tumor should be understood to include not only destruction of the tumor or carcinogenic cells, inhibition of or interference with its growth or maturation..

The gene delivery vehicles of the present invention may be administered by any one of several methods of administration known in the art which account for the risk of degradation of the recombinant virus in the bloodstream and such that the virus retains its structure and is capable of infecting target cells. Within one embodiment, administration may be accomplished by microinjection of the virus, alone or in a pharmaceutically suitable carrier or diluent, through a stereotactically-located pipette or syringe. Suitable locations vary with application, but include intraocular and brain injections.

Pharmaceutical carriers and diluents which are suitable for use within the present invention include, for example, water, lactose, starch, magnesium stearate, talc, gum arabic, gelatine, polyalkylene glycols (e.g., polyethylene glycol), and the like. The pharmaceutical preparation may be made up in liquid form for example, as solution, emulsion, suspension and the like or in a solid form, for example as a powder and the like.

If necessary, the pharmaceutical preparations can be subjected to conventional pharmaceutical adjuvants such as preserving agents, stabilizing agents, wetting agents, salts for varying the osmotic pressure, and the like. The present pharmaceutical preparations may also contain other therapeutically valuable substances.

In another aspect of the present invention, the gene delivery vehicles described herein may be delivered by chronic infusion using any suitable method known in the art, including an osmotic minipump (Alza Corp.) or delivery through a time release or sustained release medium. Suitable time release or sustained release systems include any methods known in the art, including media such as Elvax (or see, for example, U.S. Pat. Nos. 5,015,479, 4,088,798, 4,178,361, and 4,145,408). When using chronic infusion, time release, or sustained release mechanisms, the composition may be injected into the cerebrospinal fluid via intrathecal or intraventricular injections, as well as into the brain substances and intraocular locations.

The gene delivery construct should be administered in a therapeutically effective amount. A therapeutically effective amount is that sufficient to treat the disorder. A therapeutically effective amount can be determined by in vitro experiment followed by in vivo studies. Expression of the inserted nucleic acid segment can be determined in vitro using any one of the techniques described above. Expression of the inserted nucleic acid segment can be determined in vivo using any one of several methods known in the art, including immunofluorescence using a fluoresceinated ligand.

In another aspect of the present invention, the expression cassettes or gene delivery constructs described above may be incorporated into a pharmaceutical composition. Preferably, the pharmaceutical composition contains one or more therapeutically effective doses of the cassette or construct in a suitable pharmaceutical carrier or diluent. Suitable pharmaceutical carriers and diluents are outlined above. A therapeutically effective dose may be determined by in vitro experiment followed by in vivo studies as described above. The composition may be administered by any one of the methods described above.

The following examples are provided by way of illustration, and not by way of limitation.

### EXAMPLES

Unless otherwise indicated, the specific protocols used in the following examples are described in detail in Maniatis et al., supra, or Sambrook et al., supra. Parental cells and viruses, as well as attachments assays which are utilized in a variety of experiments, are briefly described below.

Cells and Viruses. The parental L cell used was the clone 1D line of Lmtk mouse fibroblasts. The procedure for the isolation of the mutant sog9 cell line was described previously (Banfield et al., Journal of Virology 69:3290-8 (1995); Gruenheid et al., Journal of Virology 67:93-100 (1993)). L and sog9 cells were grown at 37° in DMEM supplemented with 10% FBS in a 5% CO₂ atmosphere. Sog9-EXT1 and L-EXT1 cells were generated by liposome-mediated transfection with pEXT1, followed by selection in media supplemented with 700 μg/ml G418 (Gibco BRL). Clonal versions of these cell lines were derived from individual transfected colonies. The parental CHO-K1 and mutant CHOpgsA745 cell lines (Esko et al., Proc. Natl. Acad. Sci. USA 82:3197-3201 (1985)) were a gift from J. D. Esko, and were grown in Ham's F12 media supplemented with 10% FBS. HSV-1 KOS strain was a gift from D. Coen. R8102, a mutant HSV-1 F strain which has the β-galactosidase gene inserted between UL3 and UL4, under the ICP27 promoter, was a gift from B. Roizman. R8102 displays LD50 values similar to wild type HSV-1 strain F (B. Roizman, personal communication).

Attachment assay. Confluent monolayers of mutant and control L cells (containing approximately 5×10⁵ cells) growing in 24-well dishes were rinsed with phosphate-buffered saline (PBS) and incubated for 1 h at 37° in adsorption medium (DMEM-1% BSA-20 mM HEPES [pH 7.4]). Dishes were removed from the incubator and placed on ice. The medium was removed from the monolayers, and 0.1 ml of ³⁵S-methionine (>1000 Ci/mmol, ICN) radiolabeled HSV-1 strain KOS (0.5 cpm/PFU) diluted in adsorption medium was added to the wells and incubated for 2 h on ice. Following this adsorption period, the monolayers were rinsed four times with 0.5 ml of PBS prior to solubilization in cold lysis buffer (10 mM Tris-HCl [pH 7.4], 150 mM NaCl, 1 % Nonidet P-40, 1% Na-deoxycholate). Lysates were added to scintillation vials, and the radioactivity associated with the monolayers was determined by liquid scintillation spectroscopy.

### Example 1

### Cloning of EXT cDNA, and Generation of EXT Constructs

A. Assay for clones containing EXT cDNA

EXT1 cDNA was selected from a 10-million clone HeLa cell library in pcDNA3.1 (#A550-26, Invitrogen), utilizing a novel assay developed to identify genes involved in GAG biosynthesis. Briefly, this assay is based on the ability of herpes simplex virus type I (HSV-1) to infect cells by attaching to cell surface heparan sulfate-like GAGs (WuDunn and Spear, J. Virol. 63:52-58 (1989); Herold et al., Journal of General Virology 75:1211-22 (1994); Herold et al., Journal of Virology 65:1090-8 (1991); Cai et al., Virol. 62:2596-2604 (1988)). The host cells used as targets, murine sog9 cells, are unable to synthesize GAGs, and are therefore 99.5% resistant to HSV-1 infection compared with control cells (Banfield et al., Journal of Virology 69:3290-8 (1995)).

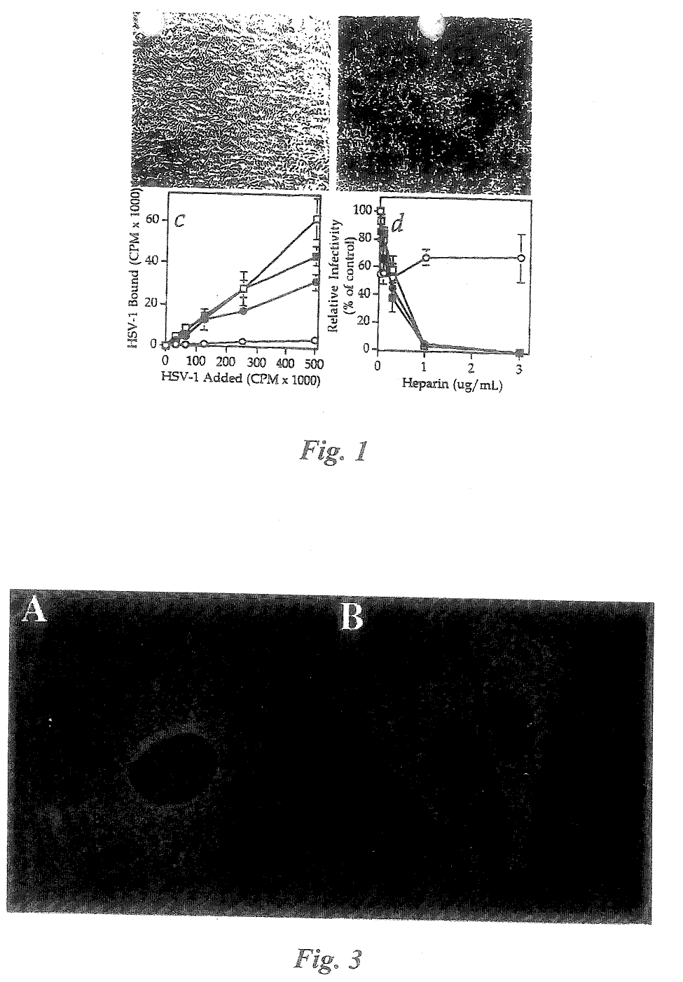

In particular, Sog9 cells were transiently transfected with pools of HeLa cell cDNAs, followed by incubation with herpes simplex virus that expresses β-galactosidase. Several cycles of screening for enhanced susceptibility to HSV-1 resulted in the isolation of EXT1, which fully restored sog9 cell infection to wild type levels (FIGS. 1a, b, c).

To test whether the rescue of HSV-1 infection conferred by EXT1 expression was the result of enhanced expression of cell surface GAG, monolayers of control L cells, sog9 cells, and EXT1-expressing sog9 cells (sog9-EXT1) were incubated at 4° C. with radiolabeled HSV-1 and the adsorbed radioactive virus was quantified by liquid scintillation spectroscopy (FIG. 1d). These assays showed clearly that EXT1 expression resulted in a partial restoration of HSV-1 attachment to sog9 cells. Competition assays (data not shown) also indicated that the attachment observed was mediated by a heparan sulfate-like molecule.

B. Generation of EXT plasmid constructs

1. pEXT1myc

pEXT1myc was constructed by PCR amplification of the EXT1 coding region using primers complementary to the translation start site (5′-CGG GAT CCC GCA GGA CAC ATG CAG GCC AAA AAA CGC TAT TTC ATC C-3′) (Sequence I.D. No. 6) and the region preceding the translation stop site (5′-TTT TCC TTT TGC GGC CGC TTT TTT CCT TAA GTC GCT CAA TGT CTC GGT A-3′) (Sequence I.D. No. 7), which contained BamHI and NotI restriction enzyme sites, respectively.

2. pΔNTMEXT1myc

pΔNTMEXT1myc was constructed using a forward primer complementary to the region of EXT1 following the putative transmembrane domain (5′-CGG GAT CCC GCA CAT GCA GTT TAG GGC ATC GAG GAG CCA CAG-3′) (Sequence I.D. No. 8), and the same reverse primer used for pEXT1myc. A translation start codon, ATG, follows the BamHI restriction enzyme site in the forward primer.

3. pΔ620EXT1myc

pΔ620EXT1myc as constructed using a reverse primer (5′-TCC CCG CGG GGA GAT TTT CTC CCC TTT TTG CTG-3′) (Sequence I.D. No. 9) containing a NotI site, and the same forward primer used for pEXT1myc. Following digestion with BamHI and NotI, the EXT1myc, ΔNTMEXT1myc and Δ620EXT1myc PCR products were ligated into pcDNA3.1/Myc-His B (#V800-20, Invitrogen), such that the myc epitope tag and 6XHis tag were in frame for subsequent translation.

4. pG339DEXT1myc and pR340CEXT1myc

pG339DEXT1myc and pR340CEXT1myc were constructed by PCR amplification using the same forward primer used for pEXT1myc, and mutagenic reverse primers which cause a G to A transition ( G339D: 5′-CAA AGC CTC CAG GAA TCT GAA GGA CCC AAG CCT GCG ATC ACG AGG AAC CAG-3′) (Sequence I.D. No. 10) or a C to T transition (R340C: 5′-CAA AGC CTC CAG GAA TCT GAA GGA CCC AAG CCT GCA ACC ACG AGG AAC CAG-3′) (Sequence I.D. No. 11). The PCR products and pEXT1myc were digested with BamHI and PpuMI (a natural restriction site adjacent to the mutations), and the PCR products were ligated into BamHI/PpuMI digested pEXT1myc. The mutated regions of EXT1 were confirmed by DNA sequencing. All primers and restriction enzymes (except PpuMI, New England Biolabs) were obtained from Gibco BRL.

### Example 2

### Glycosaminoglycan Synthesis

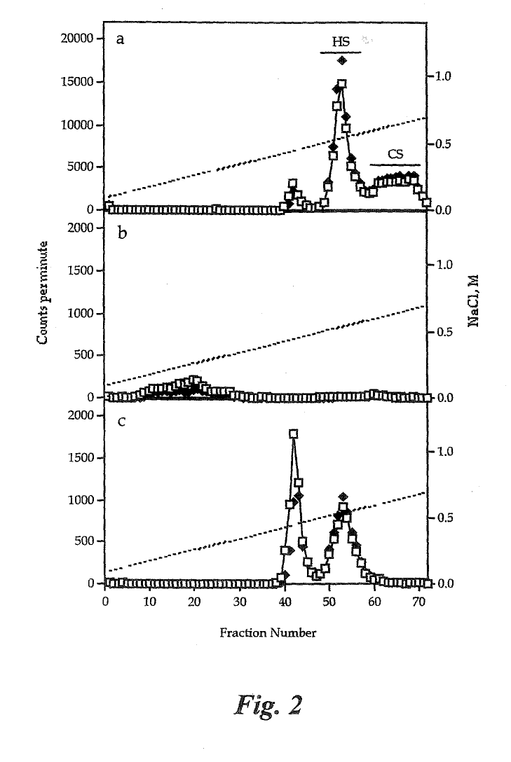

To test directly whether EXT1 expression altered the synthesis of GAGs in sog9 cells, radiolabeled GAGs were isolated from total cell extracts and analyzed by anion exchange HPLC (FIG. 2).

A. Methods

Biochemical labeling of GAGs was performed by a modification of procedures described previously by Bame and Esko (Bame and Esko, J. Biol. Chem. 264:8059-8065 (1989)). Briefly, GAGs were radiolabeled by incubation of cells for 3 days with 30 μCi of [³⁵S]sulfate (carrier free, ˜43 Ci/mg, ICN) per ml and 30 μCi of D-[6-³H]glucosamine (25-40 Ci/mmol, ICN) per ml in DMEM-FBS modified to contain 10 μM sulfate and 1 mM glucose. The cells were washed three times with cold PBS and solubilized with 1.5 ml of 0.1 M NaOH at 25° C. for 15 min. Extracts were adjusted to pH 5.5 by the addition of concentrated acetic acid and treated with 2 mg of protease (Sigma) per ml in 0.32 M NaCl-40 mM sodium acetate, pH 5.5, containing shark cartilage chondroitin sulfate (2 mg/ml) as carrier, at 37° C. for 12 hours. For some experiments, portions of the radioactive material were treated for 12 h at 37° C. with 10 mU of chondroitin ABC lyase (Sigma) or 0.5 U of heparitinase (Sigma). The radioactive products were quantified by chromatography on DEAE-Sephacel (Pharmacia) by binding in 50 mM NaCl followed by elution with 1 M NaCl. For HPLC analysis, the GAG samples were desalted by precipitation with ethanol. Following centrifugation. the ethanol precipitates were suspended in 20 mM Tris, pH 7.4, and resolved by anion exchange HPLC with a TSK DEAE-3SW column (15 by 75 mm; Beckman Instruments). Proteoglycans were eluted from the column with a linear 50 to 700 mM NaCl gradient formed in 10 mM KH₂PO4 (pH 6.0). All buffers contained 0.2% Zwittergent 3-12 (Calbiochem) to extend the life of the column. The GAGs in the peaks were identified by digestion of the sample with the relevant enzymes prior to chromatography.

B. Results

Control L cells synthesized two major sulfated peaks representing heparan sulfate (HS) (fractions 49-57) and chondroitin sulfate (CS) (fractions 60-70) (FIG. 2a). An additional sulfated peak (fractions 40-45) was also evident. As shown previously (Banfield, Journal of Virology 69:3290-8 (1995)), sog9 cells have lost the ability to synthesize any of the major GAG species (FIG. 2b). By contrast, EXT1 expression caused sog9 cells to synthesize two forms of GAG (FIG. 2c). One GAG peak (fractions 49-57) corresponded to the elution position of HS, while the other peak at fractions 40-45 was unidentified. The peak which eluted between fractions 49-57 in sog9-EXT1 cells was sensitive to digestion with heparitinase confirming its identity as a HS-like GAG (data not shown).

We also investigated whether EXT1 altered GAG expression in a wild type L cell line. HPLC analysis of GAGs isolated from EXT1-expressing L cells (L-EXT1) showed a reduction of the major HS peak relative to untransfected L cell controls, whereas CS synthesis was relatively unaffected (data not shown). This suggested that the activity of EXT1 included the ability to reduce certain GAG moieties while inducing others. To investigate whether this led to functional alterations in the cell surface architecture, HSV-1 infection efficiency was determined in EXT1-transformed L cells and Vero cells. Two observations were significant; EXT1 expression in wild type cells increased susceptibility to HSV-1 infection by 2-3 fold, and led to a dramatic increase in the cell-to-cell spread of the virus (data not shown). These results indicated that EXT1 expression exerted a profound effect on cell surface architecture, which included altering the display of cell surface GAGs.

### Example 3

### Intracellular Localization of EXT1

A. Indirect immunofluorescence microscopy.

Monolayers of CHOpgsA745, CHO—K1, sog9 and L cells were grown on glass coverslips to 70% confluence in either Ham's F12-10% FBS or DMEM-10% FBS. Transfection of these cells with myc-tagged EXT1 constructs was carried out following the LipofectAMINE (Gibco BRL) protocol. At 24 h post-transfection, the cells were rinsed with PBS and fixed in 4% paraformaldehyde for 15 min, followed by a 15 min incubation in the blocking solution (PBS with 1% BSA). After blocking, cells were incubated with anti-myc monoclonal antibody (Invitrogen) at 1:500 in PBS-1% BSA with 0.25% Saponin (Sigma) for 1 hours. Cells were washed three times with PBS, then incubated with goat anti-mouse IgG conjugated to Texas Red (Jackson Immunochemicals) diluted 1:200 in PBS-1% BSA for 30 min. The cells were then rinsed with PBS and mounted on glass coverslips. Immunofluorescence staining was observed using a Bio-Rad MRC 600 confocal epifluorescence microscope. Confocal images were rendered using NIH Image Version 1.60 and colorized with Adobe Photoshop Version 4.0 (Adobe Systems Inc.). Standard control experiments were performed, including incubation with the secondary antibody only, and with mock infected cells. All fixation and antibody incubations were performed at room temperature.

B. Results

An analysis of the EXT1 amino acid sequence suggested that it could be a transmembrane glycoprotein that assumes a type II configuration with a seven amino acid cytoplasmic amino-terminus, a nineteen amino acid signal anchor sequence, and a long, lumenal carboxy-terminus. To test this hypothesis, an EXT1 construct containing a carboxy-terminal myc-epitope tag and a truncated 5′ non-coding region, was generated as described in Example 1 (EXT1myc, FIG. 5). This construct retained 84±10% of the activity of the unmodified EXT1, which indicated that the myc tag did not seriously impede the function of this protein, and that the upstream non-coding region was not required for expression.

To investigate the subcellular localization of EXT1, cell monolayers were transfected with EXT1myc and analyzed by indirect immunofluorescence using mouse anti-myc MAb followed by Texas red conjugated goat anti-mouse IgG. Confocal micrographs (FIG. 3a) revealed a staining pattern characteristic of the endoplasmic reticulum. This was confirmed by double staining for the ER-resident protein calnexin (data not shown). This pattern was detected in all cell types investigated.

The identification of EXT1 as an ER-resident protein suggested that it exerted its effects on the cell surface by altering proteins traversing the secretory organelles en route to the cell surface. To investigate this further, a construct was generated in which the amino-terminal 26 amino acids were deleted from the EXT1myc construct (ΔNTMEXT1myc, FIG. 5). Because these 26 amino acids contained the putative signal anchor sequence, we reasoned that ΔNTMEXT1myc should not be translocated to the ER upon translation. Following transfection of ΔNTMEXT1myc, the N-terminally deleted form of EXT1 was found in a diffuse cytoplasmic location (FIG. 3b), and did not function in the HSV-1 infection assays (FIG. 5). These results indicated that the amino terminal hydrophobic domain was important for EXT1 localization and function.

To identify the minimal domain of EXT1 responsible for ER retention/retrieval, a construct was made in which the carboxy-terminal 620 amino acids of EXT1 were deleted from the EXT1myc construct (Δ620EXT1myc; FIG. 5). Although this EXT1 construct had no activity in the functional HSV-1 assays, it did assume an ER localization (FIG. 5). Thus, the amino terminal 126 amino acids of EXT1 were sufficient for ER retention/retrieval.

### Example 4

### Immunoblot Analysis

A. Methods

Immunoblots. Cell lines were transfected with myc tagged EXT1 constructs using LipofectAMINE. After 30 h, cells were washed three times with PBS and lysed in 60 mM n-octyl-b-glucopyranoside-TNE (10 mM Tris pH 7.5, 150 mM NaCl, and 2 mM EDTA) containing 1 μg/ml aprotinin (Boehringer Mannheim) and 1 μg/ml leupeptin (Boehringer Mannheim) and incubated on ice for 10 min. Lysates were clarified by centrifugation at 8000Xg at 4° C. for 10 min. Portions of the lysates were digested with 1 mU endoH (Boehringer Mannheim), or mock digested in endoH buffer (85 mM sodium citrate-1 μg/ml aprotinin-1 ug/ml leupeptin). Lysates were resuspended in an equal volume of 2X sample buffer, boiled and separated on 7.5% polyacrylamide gels, and transferred to PVDF Immobilon-P membrane (Millipore). Membranes were incubated with a 1/5000 dilution of mouse anti-myc Mab (Invitrogen) or 1 μg/ml of 9E10, a mouse anti-myc MAb (ATCC) in TBST (0.1% Tween-20, 20 mM Tris pH 7.5, 150 mM NaCl) and 1%BSA for 3 hs, washed, and incubated with a 1/10,000 dilution of horseradish peroxidase (HRP)-conjugated goat anti-mouse MAb in TBST for 1 hours. Membranes were washed thoroughly and developed using the chemiluminescence assay according to the manufacturer's instructions (KPL LumiGLO Chemiluminescent Substrate Kit). Prestained molecular weight markers (Bio-Rad) were run on gels and transferred to the PVDF membrane.

B. Results

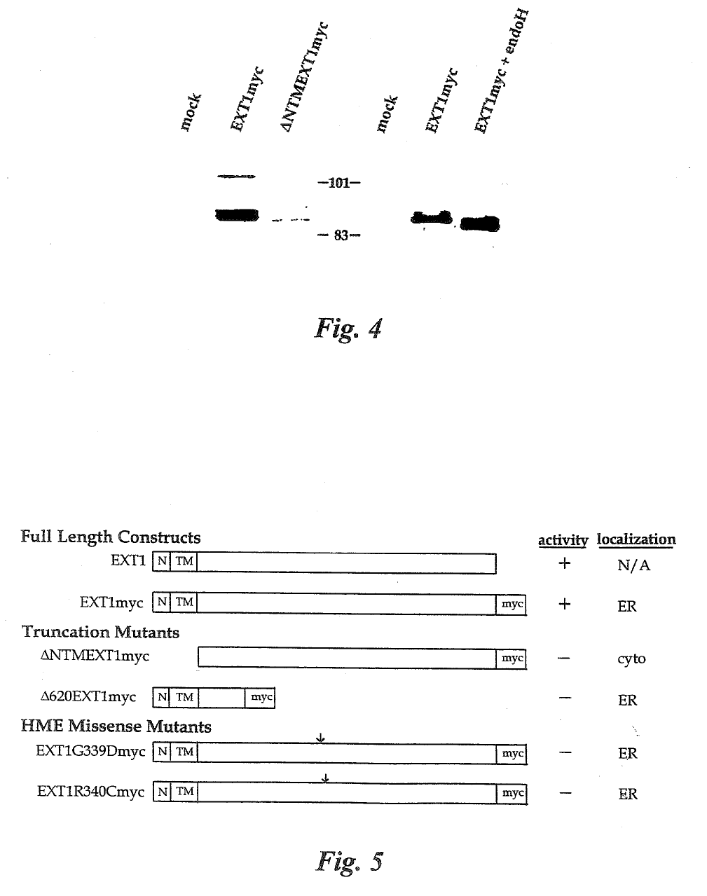

Western blots of EXT1myc isolated from transfected Chinese hamster ovary (CHO) cells revealed two forms of EXT1myc: 91 and 88 kDa (FIG. 4). By contrast, the polypeptide expressed from the ΔNTMEXT1myc construct accumulated as a single band of 88 kDa. Because EXT1 has two consensus addition sites for N-linked oligosaccharides, it was likely that the 91 kDa and 88 kDa forms of EXT1myc represented alternatively processed forms of the ER localized protein. To test whether EXT1 was a glycoprotein as predicted from the sequence, proteins were extracted from EXT1myc expressing cells and incubated with endoglycosidase H (endoH) prior to immunoblotting. Endo H cleaves high mannose N-linked oligosaccharide moieties from the protein backbone, and as such is an excellent reagent for determining the relative extent of N-linked processing in mammalian cells. In these experiments, endo H trimmed the high-mannose N-linked oligosaccharides from both the 91 kDa and 88 kDa species, generating 88 kDa and 85 kDa products (FIG. 4), which indicated that EXT1 was a glycoprotein modified by high mannose sugars characteristic of the ER and proximal Golgi elements. By contrast, ΔNTMEXT1myc, which was not translocated into the ER, was endoH resistant (data not shown). These data indicate that both forms of EXT1myc were glycosylated in the ER.

To further assess the ER localization of EXT1myc, CHO cells were transfected with EXT1myc followed by radiolabeling with ³⁵S-methionine for 1 hours. Excess methionine was added and the monolayers harvested at 0, 0.5, 1, 2 and 4 hs post-chase. SDS-PAGE analysis showed that even after 4 h, the majority of the radiolabeled EXT1myc remained endoH sensitive (data not shown). These data suggest that the N-linked oligosaccharides attached to EXT1myc were unable to be processed into complex forms, and remained in the high mannose forms found predominantly in the ER and cis Golgi compartments. Moreover, glycosylation at these two sites indicated that they were in the ER lumen, which is consistent with a type II membrane topology.

### Example 5

### Disease-Causing Mutations in EXT1

Constructs containing missense mutations at amino acid 339 (G339DEXT1myc) and 340 (R340CEXT1myc) were generated, transfected into sog9 cells, and subjected to the HSV-1 infection assays (FIG. 5).

In summary, both mutated EXT1 constructs were totally inactive in these assays, despite the fact that full length forms of the protein were synthesized and localized to the ER, as predicted. Taken together, these results provide strong evidence for a link between the ability of EXT1 to functionally alter GAG expression and the disease phenotype of HME. Moreover, the HSV-1 assay appears to provide a sensitive, surrogate measure for EXT1 activity that may be relevant to the disease.

### Example 6

### Insertion of a Sequence Encoding an EXT into an HSV-Based Recombinant Viral Vector

pTKSB (Smiley et al., J. Vir. 61(8):2368-77 (1987)), which contains the HSV-1 TK gene, is altered by insertion of a CMV promoter-containing fragment from the plasmid pRc/CMV (Invitrogen Corporation). This fragment represents the portion of the plasmid extending from base 209 to base 1285 and containing the CMV major immediate early promoter, a multicloning site, and a poly A addition site. The fragment is inserted into pTKSB by first digesting the plasmid with BamHI and then converting the BamHI site into a PacI site by the addition of adapter sequences. The CMV promoter is then oriented in the opposite direction to the TK promoter to reduce transcriptional interference. The resulting plasmid (pTKSB containing the CMV promoter) is then digested with EcoRI and HindIII or other suitable enzymes and ligated to the EXT1 coding sequence which had also been digested with HindIII and EcoRI (or other suitable enzymes) using conventional methods. This plasmid is referred to as pTKext1.

pTKext1 is then used to generate an HSV recombinant virus by in vivo homologous recombination. Briefly, pTKext1 is cotransfected into Vero cells (ATCC Accession No. CRL 1587) along with an infectious HSV-1, vhsA. vhsA is a mutant HSV-1 (FIG. 1a) (see Smibert and Smiley, J. Vir. 64:3882, 1990) containing the β-galactosidase gene in the UL41 gene coding sequence.

TK deficient recombinants are selected using bromodeoxycytidine. Following selection, virus isolates are plaque purified and tested for the EXT insert by digestion with EcoRI (other suitable enzyme), electrophoresis on a 1.1% agarose/TAE gel and hybridization to a radioactive probe. The probe can be generated by incubating the EXT gene in buffer containing random hexamers of DNA to act as primers for extension by DNA polymerase in the presence of dGTP, dTTP, dATP, and 100 mCi [³²P] dCTP. After 3 hours of incubation, the probe is used in hybridization at 37° C. in the presence of 50% formamide, 2X standard saline citrate, 5X Denhardt's solution, 1% sodium dodecyl sulfate. Following incubation for 12 h, filters were washed extensively in 0.2×SSC, 0.1% SDS, dried, and exposed to X-ray film until a signal was detected.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for altering glycosaminoglycan expression in cells, comprising altering expression or activity of an EXT gene within said cell and thereby altering said glycosaminoglycan expression.

2. The method according to claim 1 wherein expression of said EXT gene is altered by administering to said cells a small molecule which alters EXT gene expression.

3. The method according to claim 1 wherein expression of said EXT gene is altered by delivering a vector to said cells which directs the expression of an antisense or ribozyme molecule which inhibits expression of EXT genes.

4. The method according to claim 1 wherein expression of said EXT gene is altered by delivering a vector to said cells which directs the expression of an EXT gene.

5. The method according to claim 4 wherein said vector is a viral vector.

6. The method according to claim 3 wherein said viral vector is generated from a virus selected from the group consisting of herpes viruses, retroviruses, adenoviruses, parvoviruses, alphaviruses, and pox viruses.

7. The method according to claim 1 wherein said glycosaminoglycan is heparan sulfate.

8. The method according to claim 1 wherein said EXT gene is selected from the group consisting of EXT1 and EXT2.

9. The method according to claim 1 wherein expression of said glycosaminoglycan is increased.

10. The method according to claim 1 wherein expression of said glycosaminoglycan is decreased.

11. The method according to claim 1 wherein said cells are ex vivo.

12. The method according to claim 1 wherein said cells are in vivo.

13. A method for treating tumors, comprising administering to a patient a vector which directs the expression of an EXT gene, such that growth of said tumor is diminished.

14. A method for treating cancer, comprising administering to a patient a vector which directs the expression of an EXT gene, such that said cancer is treated.

15. The method according to claims 13 or 14 wherein said vector is a viral vector.

16. The method according to claim 15 wherein said viral vector is generated from a virus selected from the group consisting of herpes viruses, retroviruses, adenoviruses, parvoviruses, alphaviruses, and pox viruses.

17. The method according to claims 13 or 14 wherein said vector is directly administered into a tumor.

18. The method according to claims 13 or 14 wherein said vector also directs the expression of an additional tumor suppressor gene.

19. The method according to claim 18 wherein said tumor suppressor gene is p53, BRCA1, or, Rb.

20. A gene delivery vehicle which directs the expression of an EXT gene.

21. A gene delivery vehicle which directs the expression of a nucleic acid molecule which inhibits the expression of an EXT gene.

22. The gene delivery vehicle according to claim 21 wherein said nucleic acid molecule is an antisense molecule.

23. The gene delivery vehicle according to claim 21 wherein said nucleic acid molecule is a ribozyme molecule.

24. The gene delivery vehicle according to claim 20 or 21 wherein said vehicle also directs the expression of a tumor suppressor gene or an immunomodulatory cofactor.

25. The gene delivery vehicle according to claim 20 or 21 wherein said EXT gene is EXT1 or EXT2.

26. A cell that contains gene delivery vehicle according to any one of claims 20 to 25.

27. The cell according to claim 26 wherein said cell is a mammalian cell.

28. The cell according to claim 26 wherein said cell is a non-mammalian cell.

29. An assay for determining the ability of a compound to alter EXT activity, comprising introducing a compound into cell which express EXT, and determining whether said compound alters the quantity or species of glycosaminoglycan produced by the cell.

30. The assay according to claim 29 wherein said glycosaminoglycan is heparan sulfate.

31. The assay according to claim 29 wherein expression of glycosaminoglycan is determined by ELISA.

32. The assay according to claim 29 wherein expression of glycosaminoglycan is determined by assaying said cells for herpes infectivity.

33. The assay according to claim 29 wherein alteration of the ability of the cell to produce a glycosaminoglycan is determined by assessing the ability of glycosaminoglycan binding-ligands to bind to said cell.

34. The assay according to claim 29 wherein said cell is a cell membrane.

35. The assay according to claim 29 wherein said cells which express EXT are sog9 cells.

36. A method for determining EXT activity in cells, comprising:
(a) obtaining a nucleic acid molecule from a first cell which encodes EXT;
(b) expressing-said nucleic acid molecule in a second cell; and
(c) determining whether the expression of said nucleic acid molecule in said second cell alters glycosaminoglycan quantity of species of said first cell.

37. A method for identifying genes which have EXT activity, comprising expressing a nucleic acid molecule from a cell, and determining whether said cell exhibits an altered level or species of glycosaminoglycans.

38. The method according to claim 37 wherein said glycosaminoglycan is heparan sulfate.

39. The method according to claim 37 wherein the step of determining comprises assessing said cells for increased susceptibility to herpes infection.

40. The method according to claim 37 wherein the step of determining comprises measuring the ability of a heparan binding ligand to recognize said cells.

41. A transgenic non-human organism whose somatic and germ line cells contain a transgene encoding an EXT gene.

42. A transgenic non-human organisms having a phenotype characterized by cancer or tumor formation, said phenotype being conferred by a transgene contained in the somatic and germ cells of said mouse, said transgene comprising a mutant EXT gene.
